SEQUENCE LISTING (14 sequences)

SEQ ID NO: 1 - RNA, 16 nt, synthetic construct
  source 1..16: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-fluoro modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide attached to trivalent N-acetylgalactosamine
tgtgccttctcatcta

SEQ ID NO: 2 - RNA, 16 nt, synthetic construct
  source 1..16: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide attached to trivalent N-acetylgalactosamine
ggacagagttatcgaa

SEQ ID NO: 3 - RNA, 16 nt, synthetic construct
  source 1..16: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-fluoro modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide attached to trivalent N-acetylgalactosamine
ggacagagttatcgaa

SEQ ID NO: 4 - RNA, 16 nt, synthetic construct
  source 1..16: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-fluoro modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide attached to trivalent N-acetylgalactosamine
ggctccttctgaacaa

SEQ ID NO: 5 - RNA, 16 nt, synthetic construct
  source 1..16: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-fluoro modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide attached to trivalent N-acetylgalactosamine
ggctccttctgaacaa

SEQ ID NO: 6 - RNA, 16 nt, synthetic construct
  source 1..16: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-fluoro modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide attached to trivalent N-acetylgalactosamine
agaacttgccaagcta

SEQ ID NO: 7 - RNA, 17 nt, synthetic construct
  source 1..17: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-fluoro modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  misc_difference 17: note = 2'-O-methyl modified nucleotide with phosphorothioate linkage attached to Cholesterol, DL-tocopherol, Palmitic acid, Linoleic acid, Linolenic acid, Palmitic acid-Amino modifier C6 amidite, Palmitic acid-Aminoethoxyethoxyacetic acid-Amino modifier C6 amidite, Behenic acid-Amino modifier C6 amidite, Behenic acid-Aminoethoxyethoxyacetic acid-Amino modifier C6 amidite, 2x (Palmitic acid-Aminoethoxyethoxyacetic acid)-(gamma-aminobutyric acid-D-Lysine) -Amino modifier C6 amidite, 2x (Palmitic acid-Aminoethoxyethoxyacetic acid)-(gamma-aminobutyric acid-Beta-Homolysine) -Amino modifier C6 amidite, Palmitic acid/Dodecanoic acid (C12)-(gamma-aminobutyric acid-D-Lysine) -Amino modifier C6 amidite, Palmitic acid/Tridecanoic acid (C13)-(gamma-aminobutyric acid-D-Lysine) -Amino modifier C6 amidite, Palmitic acid/Myristic acid-(gamma-aminobutyric acid-D-Lysine) -Amino modifier C6 amidite,; note = CONT. FROM ABOVE: Palmitic acid/Pentadecanoic acid (C15)-(gamma-aminobutyric acid-D-Lysine) -Amino modifier C6 amidite, or trivalent N-acetylgalactosamine-(gamma-aminobutyric acid-D-Lysine-Beta-Homolysine) -Amino modifier C6 amidite
ttaatcctcactctaaa

SEQ ID NO: 8 - RNA, 23 nt, synthetic construct
  source 1..23: mol_type = other RNA
  modified_base 1: mod_base = OTHER; note = 5'-(E)-vinylphosphonate 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide with phosphorothioate linkage
  modified_base 22: mod_base = OTHER; note = 2'-O-methyl modified nucleotide with phosphorothioate linkage
  modified_base 23: mod_base = OTHER; note = 2'-O-methyl modified nucleotide
tttagagtgaggattaaaatgag

SEQ ID NO: 9 - RNA, 23 nt, synthetic construct
  source 1..23: mol_type = other RNA
  modified_base 1: mod_base = OTHER; note = Phosphorylated 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide with phosphorothioate linkage
  modified_base 22: mod_base = OTHER; note = 2'-O-methyl modified nucleotide with phosphorothioate linkage
  modified_base 23: mod_base = OTHER; note = 2'-O-methyl modified nucleotide
tttagagtgaggattaaaatgag

SEQ ID NO: 10 - RNA, 19 nt, synthetic construct
  source 1..19: mol_type = other RNA
  modified_base 1: mod_base = OTHER; note = Phosphorylated 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide with phosphorothioate linkage
  modified_base 16: mod_base = OTHER; note = 2'-fluoro modified nucleotide with phosphorothioate linkage
  modified_base 17: mod_base = OTHER; note = 2'-O-methyl modified nucleotide with phosphorothioate linkage
  modified_base 18: mod_base = OTHER; note = 2'-O-methyl modified nucleotide with phosphorothioate linkage
  modified_base 19: mod_base = OTHER; note = 2'-O-methyl modified nucleotide
taagagaaaactggtcaga

SEQ ID NO: 11 - RNA, 19 nt, synthetic construct
  source 1..19: mol_type = other RNA
  modified_base 1: mod_base = OTHER; note = Phosphorylated 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide with phosphorothioate linkage
  modified_base 16: mod_base = OTHER; note = 2'-fluoro modified nucleotide with phosphorothioate linkage
  modified_base 17: mod_base = OTHER; note = 2'-O-methyl modified nucleotide with phosphorothioate linkage
  modified_base 18: mod_base = OTHER; note = 2'-O-methyl modified nucleotide with phosphorothioate linkage
  modified_base 19: mod_base = OTHER; note = 2'-O-methyl modified nucleotide
tatcaggagtgactgaaat

SEQ ID NO: 12 - RNA, 20 nt, synthetic construct
  source 1..20: mol_type = other RNA
  modified_base 1: mod_base = OTHER; note = 5'-(E)-vinylphosphonate 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide with phosphorothioate linkage
  modified_base 18: mod_base = OTHER; note = 2'-O-methyl modified nucleotide with phosphorothioate linkage
  modified_base 19: mod_base = OTHER; note = 2'-O-methyl modified nucleotide with phosphorothioate linkage
  modified_base 20: mod_base = OTHER; note = 2'-O-methyl modified nucleotide
taaaggtgcaaacatgtaac

SEQ ID NO: 13 - RNA, 16 nt, synthetic construct
  source 1..16: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-fluoro modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide attached to trivalent N-acetylgalactosamine
gcagtatgttgatgga

SEQ ID NO: 14 - RNA, 16 nt, synthetic construct
  source 1..16: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-fluoro modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide attached to trivalent N-acetylgalactosamine
tgtgccttctcatcta

(19) United States
(12) Patent Application Publication
(10) Pub. No.: US 2024/0327448 A1
(43) Pub. Date: Oct. 3, 2024

(54) LITHIUM REAGENTS AND THEIR USES IN SOLID PHASE SYNTHESIS

(71) Applicant: OliX US, Inc., San Diego, CA (US)

(72) Inventors: Dongwon Shin, San Diego, CA (US); Namho Kim, San Diego, CA (US); Jongsu Yoo, San Diego, CA (US)

(21) Appl. No.: 18/443,778

(22) Filed: Feb. 16, 2024

Related U.S. Application Data
(60) Provisional application No. 63/446,270, filed on Feb. 16, 2023.
(60) Provisional application No. 63/446,271, filed on Feb. 16, 2023.

(51) Int. Cl. C07H 21/00

(52) U.S. Cl. CPC C07H 21/00

(57) ABSTRACT

Provided herein are lithium-including reagents useful in solid phase synthesis. Provided herein are improved methods of oligonucleotide synthesis on UnySupport solid support.

### CROSS REFERENCE TO RELATED APPLICATIONS

This application claims the benefit of U.S. Provisional Application No. 63/446,270 filed Feb. 16, 2023, and U.S. Provisional Application No. 63/446,271 filed Feb. 16, 2023, which are incorporated herein by reference in their entirety.

### SEQUENCE LISTING

The instant application contains a Sequence Listing which has been submitted electronically in XML format and is hereby incorporated by reference in its entirety. Said XML copy, created on Feb. 13, 2024, is named 0817444_00036_SL.xml and is 155,641 bytes in size.

### FIELD

The present disclosure relates to lithium assisted cleavage and deprotection methods, and related reagents, for preparing molecules on solid support. In some embodiments, the molecule includes a bioconjugated oligonucleotide.

### BACKGROUND

Oligonucleotides have been used to modulate gene expression via various processes in a wide range of diseases. A major obstacle preventing widespread usage of oligonucleotide therapeutics is the difficulty in achieving efficient delivery to target organs and tissues, and the difficulty in increasing the stability of oligonucleotides to maximize their potencies.

GalNAc conjugation enables the targeted delivery of oligonucleotides to liver and leads to the several approvals of new therapeutics from regulatory authorities. AM Chemicals developed a new GalNAc phosphoramidite utilizing piperidine and 1,3-diol compartments and commercialized it to the market. Oligonucleotide synthesis typically includes 4 steps (deblocking, coupling, oxidation/sulfurization, and capping) and utilizes a variety of solid supports. A representative solid support is UnySupport, developed by Isis Pharmaceuticals. UnySupport comprises a rigid bicyclic molecule on the support to get anchimeric assistance, which facilitates the formation of the cyclic phosphate transition state, leading to the faster cleavage of oligonucleotide from a solid support platform. An initial three consecutive oligonucleotide synthesis cycle on solid support is one of the easiest ways to introduce the tri-GalNAc (1+1+1) conjugation linker at 3′-position of oligonucleotide, called by 3′-tri-GalNAc conjugated oligonucleotide. There is interest in RNA interference (RNAi)-based therapeutics, triggered by the commercial success from Alnylam Pharmaceuticals. Oligonucleotide therapeutics that regulate gene expression have been developed towards clinical use at a steady pace and are now being used to successfully treat diseases and shows the potential for widespread application. To date, there are several marketed products based on antisense oligonucleotide (ASO), aptamers, small interfering RNAs (siRNAs), and mRNA vaccines, and many others are in the pipeline for both academia and industry. This recent progress has been triggered by the rapid development in the area of oligonucleotide modifications to improve the stability and targeted delivery.

Typical commercial processes of oligonucleotide synthesis utilize non-nucleosidic modifier phosphoramidites for the introduction of functional and reporter groups containing various ligands such as TFA (trifluoroacetyl)-amino, alkyne, fluorescein, biotin, pyrene, dabsyl and dabcyl moieties. Commercial non-nucleosidic modifier phosphoramidites for targeted cellular uptake of oligonucleotides include, for example, GalNAc (N-acetylgalactosamine), cholesteryl, DL-tocopherol, palmitic acid, behenic acid, linoleic acid, and/or linolenic acid moieties. These modifiers can be introduced to sense strands during the forward oligonucleotide synthesis for RNAi-based therapeutics (www.amchemicals.com/products).

Bioconjugated oligonucleotides, especially, with the multivalent GalNAc conjugation utilizing the cluster or monomer-based linear conjugation, have paved the way for oligonucleotide pharmaceutical development. Multivalent GalNAc allow the oligonucleotide to be delivered to the liver with high efficiency. The representative examples are the cluster conjugation of tri-antennary GalNAc from Alnylam Pharmaceuticals and monomer-based linear conjugation of non-nucleoside phosphate from AM Chemicals. Oligonucleotides containing cholesterol, DL-tocopherol, saturated or unsaturated fatty acids have been studied in a wide range of therapeutic areas such as pulmonology, ophthalmology, neurology, and/or orphan diseases. These modifications are known to utilize the hydrophobic interaction with cell membranes to enhance the cellular uptake.

For the antisense strands, the 5′-phosphate is believed to be critical for the effective loading into the Argonaute (Ago2) protein of the RNA-induced silencing complex (RISC) to elicit gene silencing. The natural 5′-phosphate is usually introduced by utilizing a chemical phosphorylation reagent such as solid CPR II. However, 5′-phosphate is rapidly hydrolyzed by phosphatases before the siRNA reaches the RISC. Among the several phosphatase-stable phosphate mimics, the (E)-vinylphosphonate (EVP) has been the most effective bioisostere of natural 5′-monophosphate and endowed the oligonucleotides with enhanced metabolic stability and efficient in vivo potency. RNAi-based therapeutics have been utilizing the duplexes by combinations of bioconjugated sense strands and antisense strands. Bioconjugated oligonucleotides are typically synthesized on solid support (i.e., UnySupport™ CPG) by utilizing the non-nucleosidic modifier phosphoramidite for the targeted cellular uptake and/or the introduction of functional and reporter groups from AM Chemicals. General cleavage and deprotection usually takes a long time or multiple steps to remove the UnySupport tether and causes the formation of various impurities. There is a need for improved methods for synthesis of multivalent conjugated oligonucleotides, e.g., multivalent GalNAc conjugated oligonucleotides, using UnySupport Solid Support.

### SUMMARY

Provided herein are lithium-including reagents useful in solid phase synthesis. It has been discovered that lithium permits efficient cleavage of a solid support bound compound where the compound on cleavage from the solid support results in an alcohol on the compound at the cleavage site. Lithium assisted cleavage and deprotection methods reduced the reaction time and quenching steps and showed a reduction of nucleophile-driven by-products. Moreover, this method was found to extend to all oligonucleotides containing 3′- and/or 5′-modifications.

During the synthesis of 3′-Tri-GalNAc conjugated oligonucleotide on UnySupport solid support (UnySupport CPG, Glen Research), the tricyclic moiety of UnySupport on tri-GalNAc conjugated oligonucleotide (UnySupport hereinafter) was not easily removed under the general cleavage and deprotection method such as ammonia gas phase or aqueous methyl amine and ammonium hydroxide liquid phase at elevated temperature. Also described herein are methods for removal of UnySupport through two-step cleavage and deprotection methods with basic and acidic reagents. A high purity of tri-GalNAc conjugated oligonucleotide was obtained through anion-exchange purification, desalting and final purification/isolation process.

### BRIEF DESCRIPTION OF THE DRAWINGS

FIG. 1a. Structure of 3′-tri-GalNAc bioconjugated oligonucleotide (SEQ ID NO: 1), FIG. 1b. Deconvoluted mass spectrum of crude product after the first basic reagent treatment during the cleavage and deprotection at 6 hours. FIG. 1c. Deconvoluted mass spectrum of crude product after the second acidic reagent treatment during the cleavage and deprotection at 0.5 hours. FIG. 1d. Deconvoluted mass spectrum of crude product after the second acidic reagent treatment during the cleavage and deprotection at 1 hour. FIG. 1e. Deconvoluted mass spectrum of crude product after the second acidic reagent treatment during the cleavage and deprotection at 6 hours.

FIG. 2a. AEX-HPLC for tri-GalNAc bioconjugated oligonucleotide (SEQ ID NO: 1) after two-step cleavage and deprotection. FIG. 2b. Expanded view of a section of the AEX-HPLC chromatogram for tri-GalNAc bioconjugated oligonucleotide (SEQ ID NO: 1) after two-step cleavage and deprotection. FIG. 2c shows lithium assisted cleavage where cleavage of a compound from the solid support results in an alcohol on the compound at the cleavage site. FIG. 2d-1. Impurity profile for the lithium assisted cleavage and deprotection method for tri-GalNAc bioconjugated oligonucleotide (SEQ ID NO: 1). FIG. 2d-2. Expanded view of a section impurity profile chromatogram. FIG. 2e-1. Impurity profile for two-step cleavage and deprotection FIG. 2e-2. Impurity profile for one-step lithium assisted cleavage and deprotection

FIG. 3. Non-nucleosidic GalNAc phosphoramidite and solid support from AM Chemicals. TMT refers to trimethoxy trityl protecting group.

FIG. 4. Conjugation of Tri-GalNAc (1+1+1) conjugation on oligonucleotide, demonstrated by AM Chemicals

FIG. 5. Simple GalNAc phosphoramidite, reported by K. G. Rajeev et al. (ChemBioChem, 2015, 16, 903). DMT refers to dimethox trityl protecting group.

FIG. 6. Synbase™-based dR-GalNAc (alpha) phosphoramidite and CPG, marketed by Biosearch Technologies.

FIG. 7. Oligonucleotide synthesis cycle, and cleavage and deprotection (C&D) process

FIG. 8. Universal support structures

FIG. 9. Type 1 mechanism: cleavage followed by deprotection and dephosphorylation

FIG. 10. Type 2 mechanism: dephosphorylation followed by cleavage and deprotection

FIG. 11. Synthetic pathway of 3′-multivalent GalNAc conjugated oligonucleotides

FIG. 12. LCMS of SEQ ID NO: 14 after cleavage and deprotection with 28%-30% ammonium hydroxide/40% aqueous methylamine (1:1 v/v) (AMA) treatment at RT for 6 hours

FIG. 13. LCMS of SEQ ID NO: 14 after cleavage and deprotection with trifluoro acetic acid (TFA) treatment at room temperature (RT) for 6 hours

FIG. 14. Anion Exchange High Pressure Liquid Chromatography (AEX-HPLC) of SEQ ID NO: 14 after AEX-HPLC purification

FIG. 15. AEX-HPLC of SEQ ID NO: 14 after desalting, filtration, lyophilization/drying under vacuum.

FIG. 16. Deconvoluted HT of SEQ ID NO:13 after cleavage and deprotection with AMA treatment at RT for 6 hours

FIG. 17. Change of deconvoluted HT of SEQ ID NO: 13 after cleavage and deprotection with TFA treatment at RT for 0.5 hours

FIG. 18. Change of deconvoluted HT of SEQ ID NO: 13 after cleavage and deprotection with TFA treatment at RT for 1.0 hours

FIG. 19. Change of deconvoluted HT of SEQ ID NO: 13 after cleavage and deprotection with TFA treatment at RT for 3.0 hours

FIG. 20. Change of deconvoluted HT of SEQ ID NO: 13 after cleavage and deprotection with TFA treatment at RT for 6.0 hours

FIG. 21. AEX-HPLC of SEQ ID NO: 13 after AEX-HPLC purification

FIG. 22. AEX-HPLC of SEQ ID NO: 13 after desalting, filtration and lyphilization/drying under vacuum.

### DETAILED DESCRIPTION

Most oligonucleotide synthesis is conducted by the nucleoside phosphoramidite method for the introduction of naturally occurring nucleosides or non-nucleosides, which is decorated for the various functionalities. In general, oligonucleotide forward synthesis is carried out by a stepwise addition of nucleoside residue to the 5′-terminus of the growing chain until the desired sequence is assembled. Each addition is referred to as a synthetic cycle composed of 4 chemical reactions: (1) deblock (detritylation), (2) coupling of amidite, (3) oxidation to P(V) state of phosphate or phosphorothioate, and (4) capping, where the order of oxidation and capping step might be changeable. On the other hand, oligonucleotides can be synthesized from 5′-terminus to 3′-terminus, namely, in oligonucleotide reverse synthesis to offer the facile approach to 3′-modification of oligonucleotides. Synthetic cycle is the same as the oligonucleotide forward synthesis, different by a stepwise addition of nucleoside residue to the 3′-terminus of the growing chain, where the 5′-hydroxyl group of first nucleoside is linked through phosphate on solid support.

After completion of oligonucleotide forward or reverse synthesis (including post-synthetic modification if required), oligonucleotide bound on solid support is cleaved and all protecting groups except 2′-O-TBDMS (tert-butyldimethylsilyl) are deprotected by basic treatment such as gaseous or aqueous ammonia, aqueous or methanolic methylamine, or a mixture of those at ambient temperature (namely, cleavage and deprotection). After optionally quenching with mild buffer solution, the filtrate is finally purified and desalted to afford the targeted oligonucleotide in designated purity.

During the cleavage and deprotection, it was found that tri-GalNAc bioconjugated oligonucleotides or reversely synthesized oligonucleotides were not easily cleaved from the UnySupport under the condition of general basic cleavage and deprotection utilizing 28%-30% ammonium hydroxide/40% aqueous methylamine 1:1 v/v (AMA) or ammonium hydroxide solution even at elevated temperature up to 80° C. for a long time. Further, the harsh conditions led to formation of various unexpected impurities and made it difficult to obtain highly pure oligonucleotides.

Thus, provided herein are methods for synthesis of bioconjugated oligonucleotides and efficient cleavage and deprotection methods.

It is one object of this disclosure to provide the efficient cleavage and deprotection method for bioconjugated oligonucleotides from solid support. Bioconjugated oligonucleotides may be synthesized by forward 3′ to 5′ direction or reverse 5′ to 3′ direction, rendering the primary alcohol linked to solid support through the phosphate linkage.

It is one object of this disclosure to provide the efficient cleavage and deprotection method for bioconjugated oligonucleotides from solid support. Bioconjugated oligonucleotides may contain mono- or multivalent conjugation introduced from moieties including, and not limited to, phosphoramidites, bioconjugation-preloaded solid supports, or post-synthetic modifications.

It is one object of this disclosure to provide the efficient cleavage and deprotection method for bioconjugated oligonucleotides from solid support. Bioconjugated oligonucleotides may contain the mono- or multivalent conjugation selected from moieties including, and not limited to, GalNAc, cholesterol, DL-tocopherol, saturated or unsaturated fatty acids including, and not limited to, palmitic acid, behenic acid, linoleic acid, and/or linolenic acid.

It is one object of this disclosure to provide the efficient cleavage and deprotection method for bioconjugated oligonucleotides from solid support. Bioconjugated oligonucleotides may contain the mono- or multivalent conjugation selected from moieties including, and not limited to, TFA amino, alkyne, fluorescein, biotin, pyrene, dabsyl and dabcyl moieties or their post synthetically modified moieties.

It is one object of this disclosure to provide the efficient cleavage and deprotection method for bioconjugated oligonucleotides from solid support. Bioconjugated oligonucleotides may contain moieties including, and not limited to, pivaloyloxymethyl (POM) protected (E)-vinylphosphonate (EVP) or chemical phosphorylation reagent (CPR)-derived phosphate functional moieties.

It is one object of this disclosure to provide the efficient cleavage and deprotection method for bioconjugated oligonucleotides from solid support. Solid support may be selected from supports including, and not limited to, universal support, universal support II, universal support III, UnyLinker, or UnySupport or bioconjugation-preloaded solid supports. Bioconjugation-preloaded solid supports may contain the mono- or multivalent conjugation such as moieties including, and not limited to, GalNAc, cholesterol, DL-tocopherol, saturated or unsaturated fatty acids such as palmitic acid, behenic acid, linoleic acid, and/or linolenic acid.

It is one object of this disclosure to provide the efficient cleavage and deprotection method for bioconjugated oligonucleotides from solid support. Cleavage and deprotection method may contain the lithium cation provided from lithium compounds such as, and not limited to, lithium fluoride, lithium chloride, lithium bromide, lithium iodide, lithium hydroxide, lithium bicarbonate, lithium decarbonate, lithium hydrogen sulfate, lithium sulfate, lithium nitrate, lithium perchlorate, lithium oxide, lithium sulfide, lithium cyanide, lithium thiocyanide, lithium acetate, lithium oxalate, and/or lithium phosphate, or their hydrate form. Concentration of lithium cation can range from about 0.1 M to about 6.0 M.

It is one object of this disclosure to provide the efficient cleavage and deprotection method for bioconjugated oligonucleotides from solid support. Cleavage and deprotection method may contain the concentrated or dilute ammonia solution (0.1 to 40% aqueous ammonia solution or 0.1 to 40% alcoholic ammonia solution, where alcohol is selected from alcohols such as, and not limited to, methanol, ethanol, n-propanol, isopropanol, n-butanol, iso-butanol, and/or tert-butanol). The volume of this solution can be selected from volumes ranging from 10 μL/μmol to 5000 μL/μmol, or other suitable volumes.

It is one object of this disclosure to provide the efficient cleavage and deprotection method for bioconjugated oligonucleotides from solid support. Cleavage and deprotection method may contain 0 to 50% alkylamine. Examples of s alkylamines include, and are not limited to, monoalkyl amines such as e.g., methylamine, ethylamine, propylamine, isopropylamine, and/or ethylenediamine, dialkylamines such as e.g., dimethylamine, diethylamine, dipropylamine, diisopropylamine, morpholine, piperidine, trialkylamines such as e.g., trimethylamine (TMA), triethylamine (TEA), tripropylamine (TPA), diisopropylethylamine (DIPEA), and alkylpiperidines such as e.g., methylpiperidine, ethylpiperidine, propylpiperidine, isopropylpiperidine.

It is one object of this disclosure to provide the efficient cleavage and deprotection method for bioconjugated oligonucleotides from solid support. Cleavage and deprotection method may contain the lithium cation and/or alkylamine in aqueous or alcoholic ammonia solution. In some embodiments, a non-limiting concentration of lithium cation may range from 0.1 M to 6.0 M. In some embodiments, a non-limiting concentration of alkylamine may range from 0% to 50% w/w. In some embodiments, a non-limiting concentration of ammonia in aqueous or alcoholic solution may range from about 0.1 to 40% w/w.

It is one object of this disclosure to provide the efficient cleavage and deprotection method for bioconjugated oligonucleotides from solid support. In some embodiments, cleavage and deprotection methods can be performed at ambient temperature and/or a non-limiting range of temperature selected from about 0.1° C. to about 80° C. until the cleavage and deprotection is completeo. In some embodiments, cleavage and deprotection method can be performed in ambient reaction time and/or a non-limiting range of reaction time selected from about 0.1 hours to about 72 hours until the cleavage and deprotection is complete.

### Oligonucleotide Synthesis, Cleavage, Deprotection, and Purification

For the oligonucleotide synthesis utilizing the solid support (herein, the UnySupport), the first coupling reaction is usually performed with nucleoside amidite for the 3′-unmodified oligonucleotide synthesis to result in phosphate internucleotide linkage between UnySupport and 3′-hydroxyl group on nucleoside, where the 3′-hydroxyl group is secondary alcohol on sugar moiety of nucleoside. This phosphate linkage is readily cleaved by any basic reagents such as aqueous ammonium hydroxide, aqueous methylamine, and AMA to result in the formation of cyclophosphate with the 10 aid of a conformationally preorganized UnySupport. Scheme 1. Brief summary of oligonucleotide forward synthesis without 3′-bioconjugation

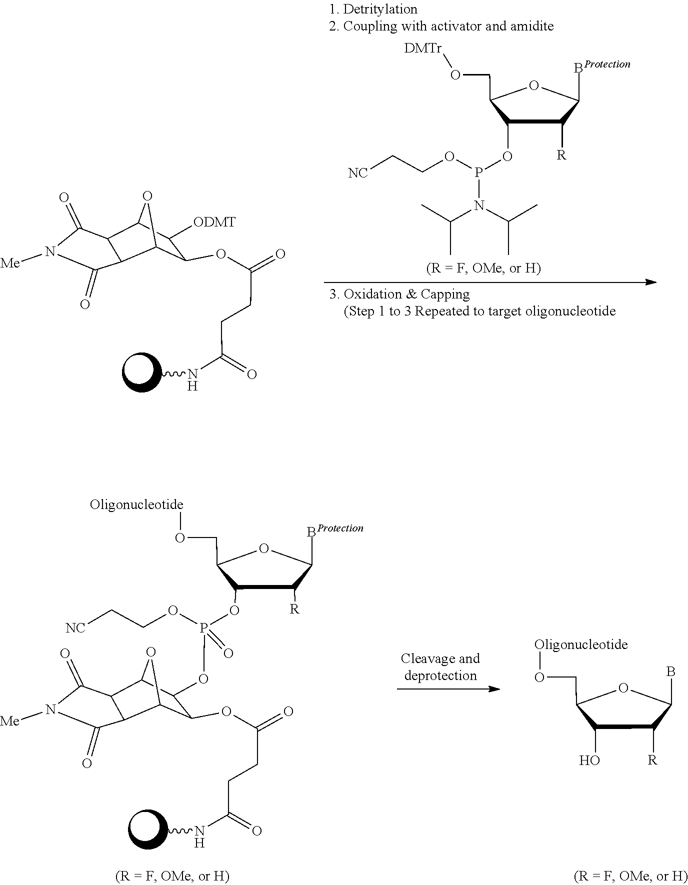

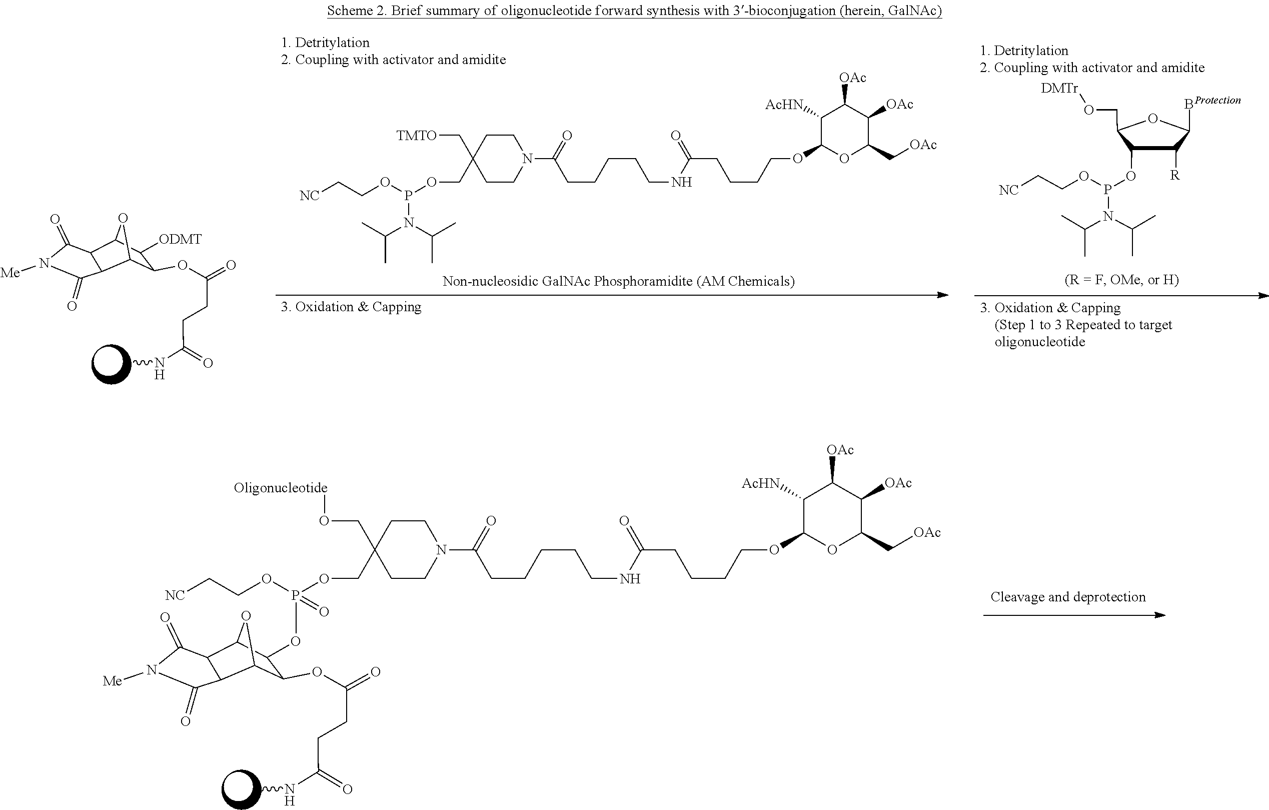

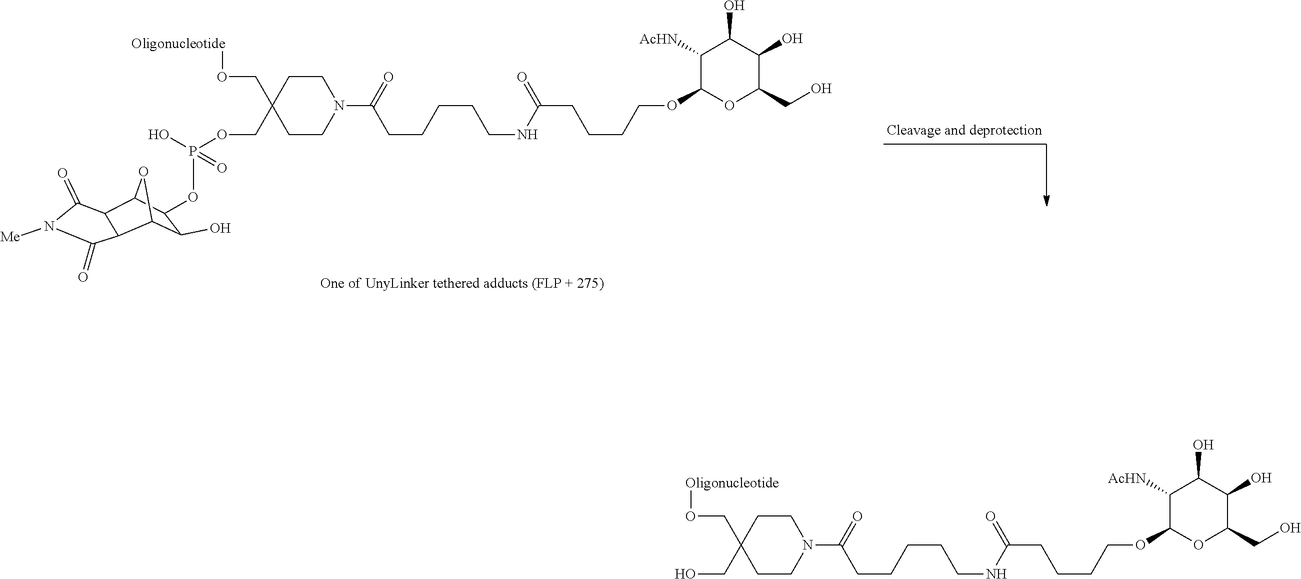

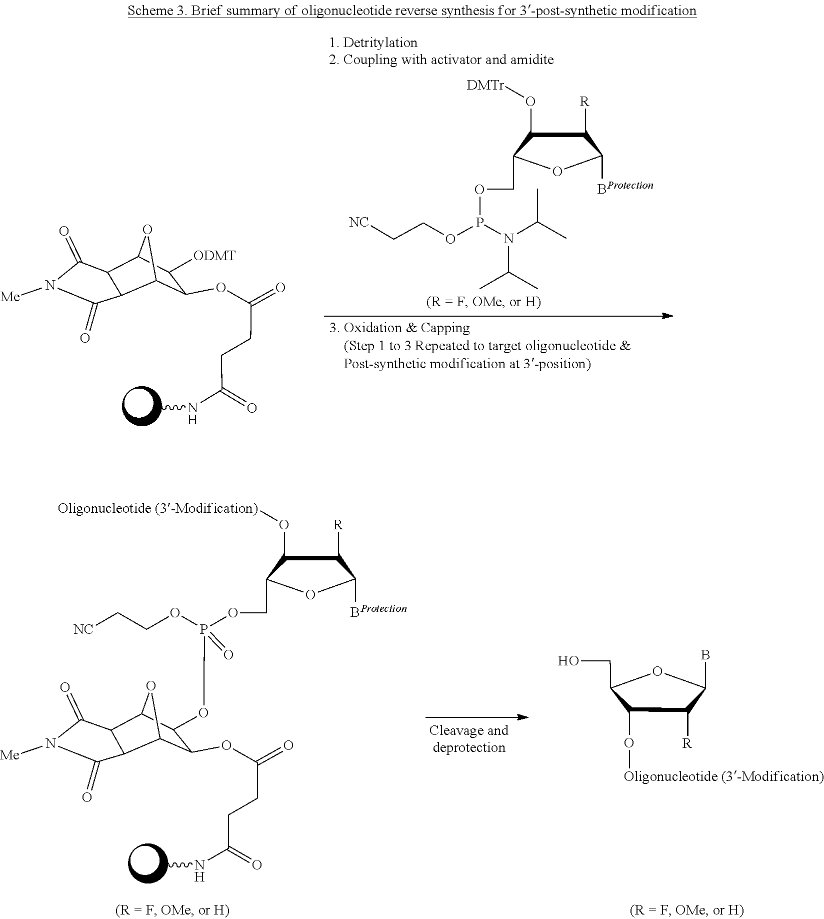

However, when the first coupling reaction is performed with non-nucleosidic phosphoramidite from AM Chemicals for the 3′-bioconjugated oligonucleotide synthesis, the resulting internucleotide phosphate linkage is formed between solid support and primary alcohol of piperidinyl conjugation linker. After basic cleavage and deprotection using AMA, the aberrant mass of oligonucleotide was observed by increased mass of m/z +275 (N-methyl pyrrolidine dione ring-closed adduct) or +293 (N-methylamido carboxylate ring-open adduct). This proved that the oligonucleotide still contained the UnySupport tether through phosphate linkage. Interestingly, this phosphate linkage is not readily cleaved by any other basic conditions, provided by AM Chemicals (U.S. Pat. No. 10,781,175 B2; Paragraph 143-144). Longer exposure under higher temperature did not show any efficient method to cleave this phosphate linkage between solid support and bioconjugation linker (Scheme 2). This phenomenon was also observed when oligonucleotide was reversely synthesized to result in the internucleotide phosphate linkage formed between solid support and 5′-primary alcohol of nucleoside (Scheme 3). Cleavage and deprotection using AMA showed the certain amount of UnySupport tether as well even at elevated temperature for extended period.

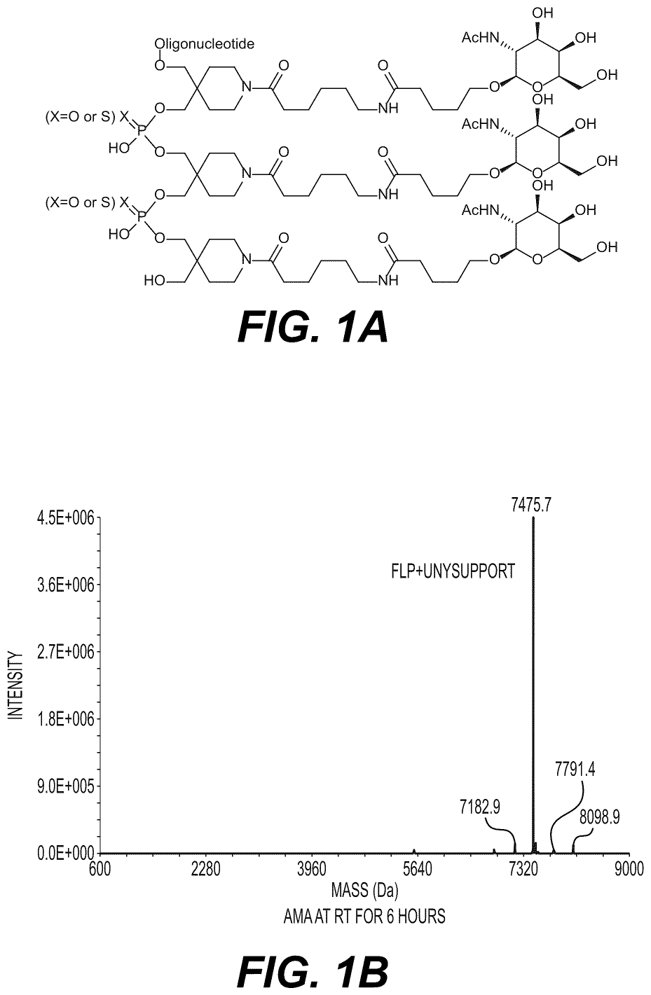

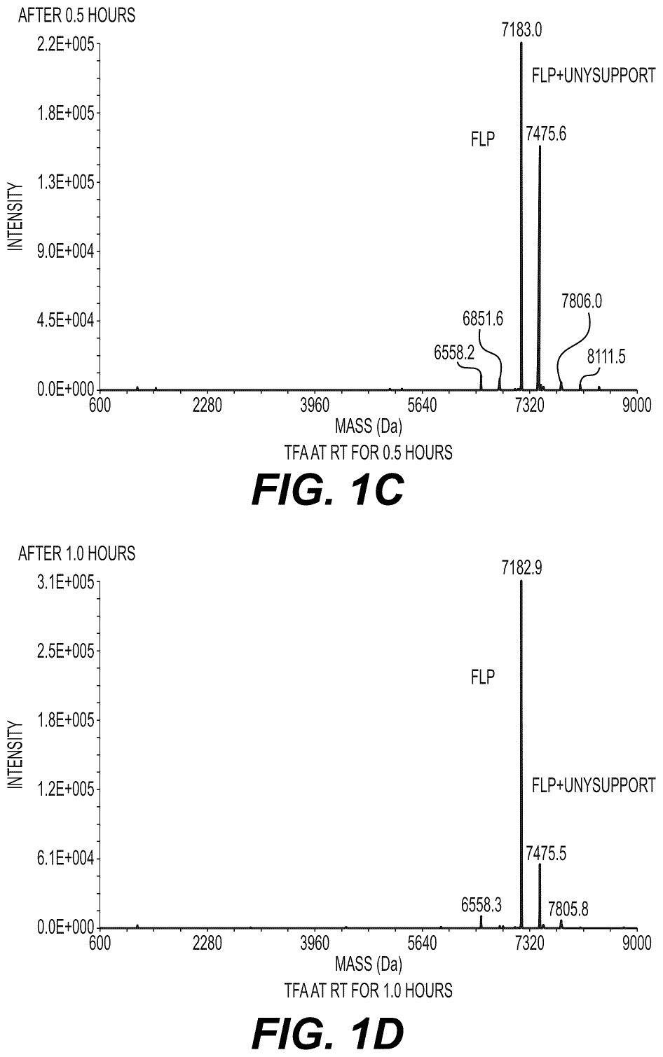

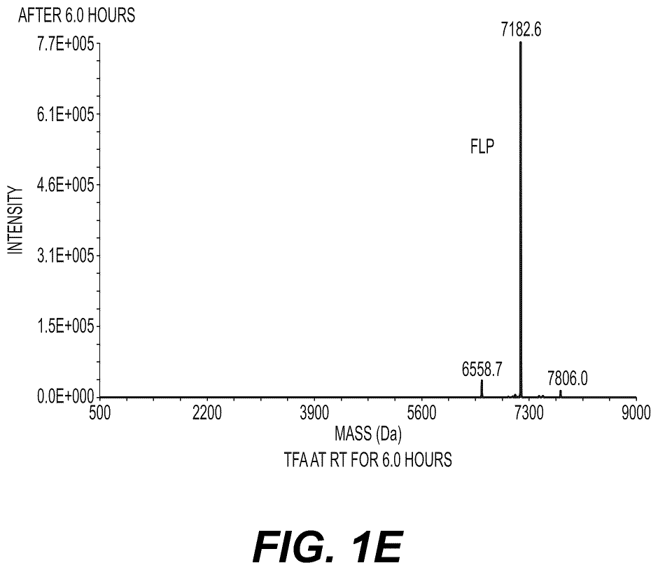

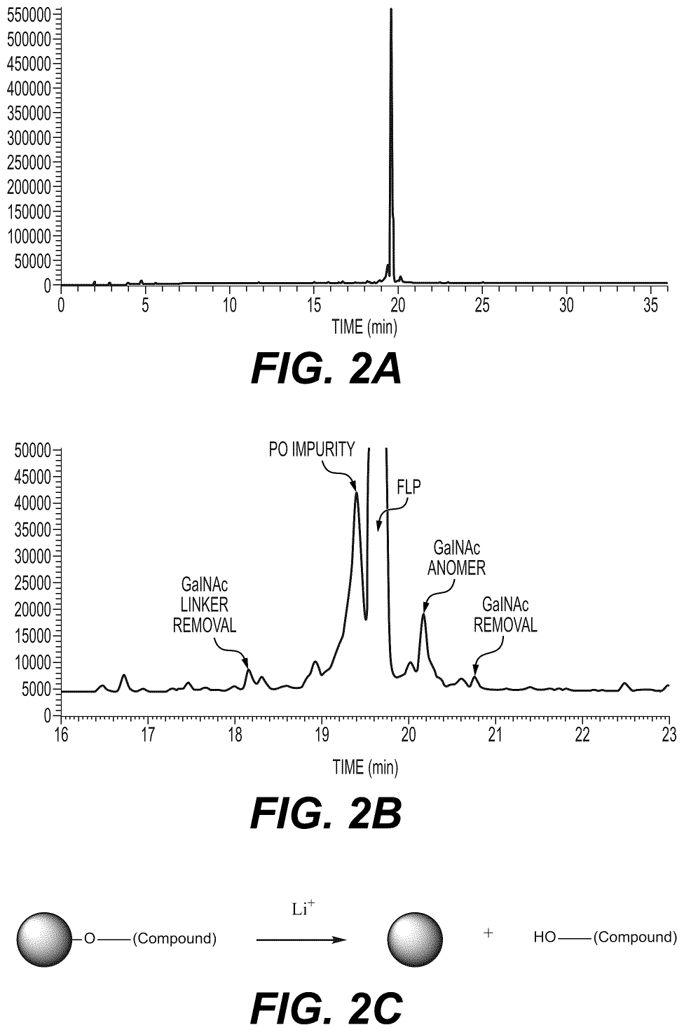

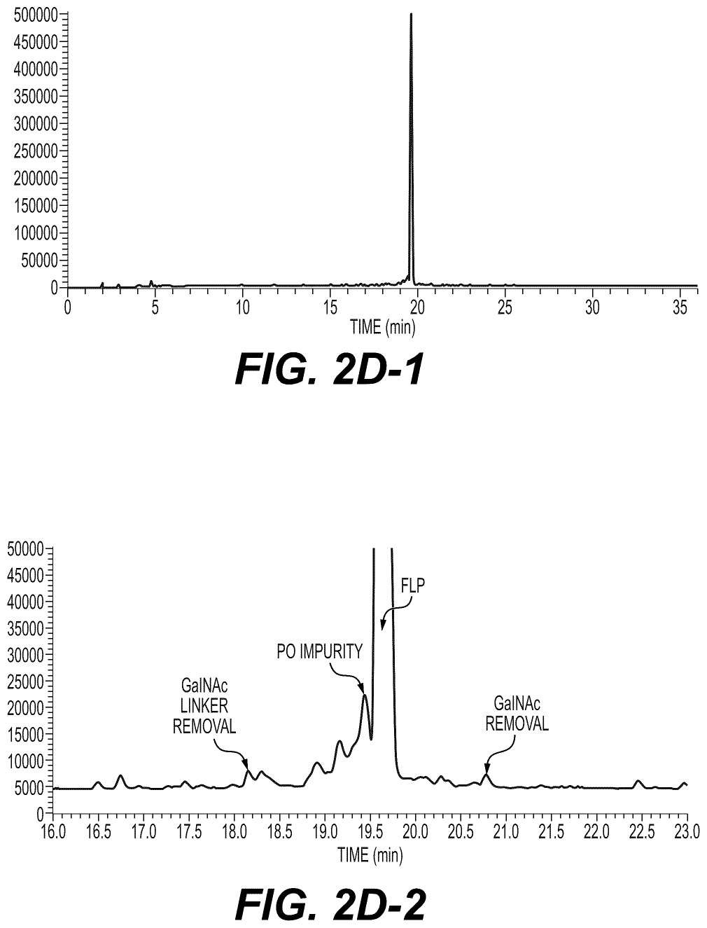

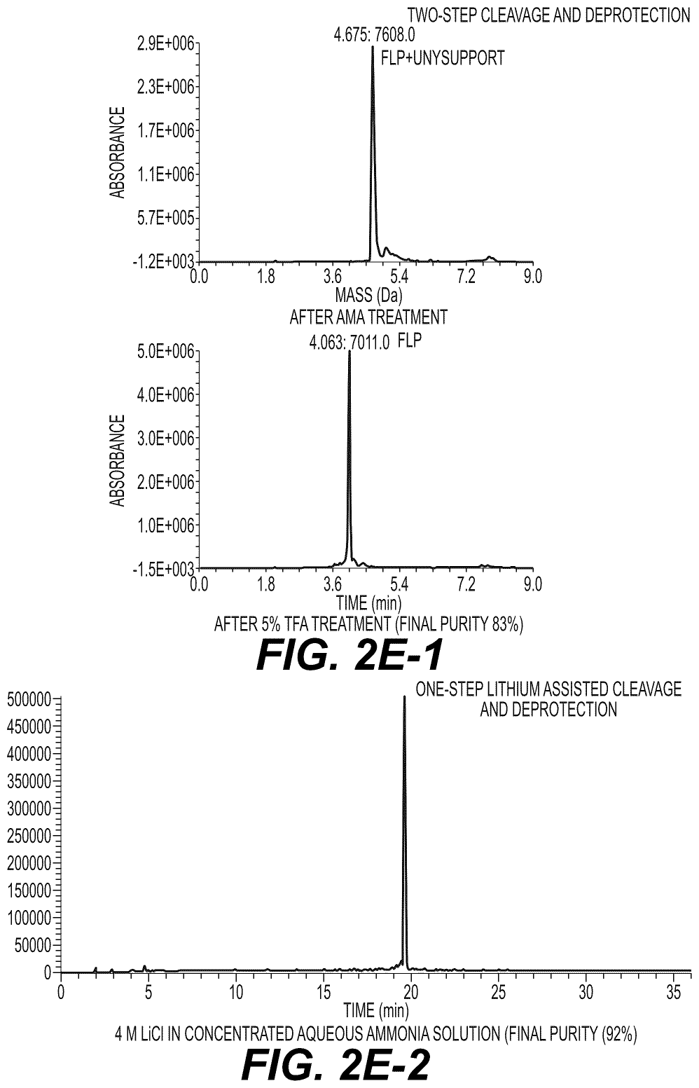

Two-step cleavage and deprotection showed the improved cleavage of UnySupport tether from bioconjugated oligonucleotides containing primary alcoholic phosphate linkage between solid support and nucleoside utilizing the AMA and aqueous trifluoroacetic acid solution. First, AMA treatment contributed to cleave the solid support linkage from bioconjugated oligonucleotide with still remaining UnySupport tether (+275 or +293 Da). Second, aqueous TFA treatment completed the cleavage of UnySupport tether from bioconjugated oligonucleotide (FIG. 1). However, it was found that the cleavage and deprotection for tri-GalNAc bioconjugated oligonucleotide showed the unexpected by-products such as GalNAc N-acetyl missing, GalNAc α/β anomer (suspected), GalNAc or GalNAc C5 linker falling-off during the two-step cleavage and deprotection process (FIG. 2).

Referring to FIG. 2b, the table below provides details for certain peaks.

| FLP and | |
|---|---|
| related | |
| impurities | Structure |
| FLP (full length product) | 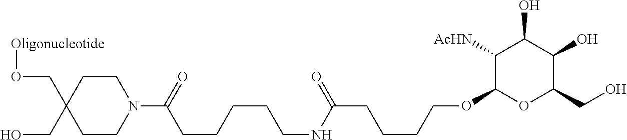 |
| GalNAc anomer (suspected) | 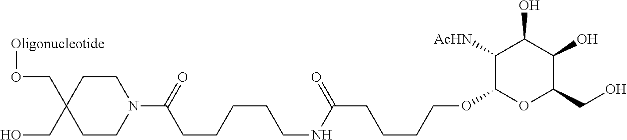 |
| GalNAc Removal (−203 Da) | 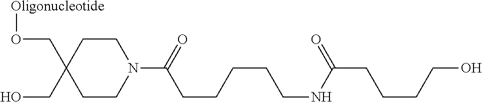 |
| GalNAc linker removal (−416 Da) | 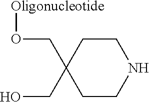 |
| PO | Phosphorothioate transformed to phosphate by sulfur-oxygen |
| Impurity | exchange |
| (−16 Da) | |

The present disclosure relates to the efficient cleavage and deprotection for bioconjugated oligonucleotides from solid support. Herein the bioconjugated oligonucleotides may be synthesized by forward 3′ to 5′ direction or reverse 5′ to 3′ direction, and in the synthesis, a primary alcohol is linked to solid support through a phosphate linkage. Herein the bioconjugated oligonucleotides may contain mono- or multivalent conjugation introduced from phosphoramidites, bioconjugation preloaded solid supports, or post-synthetic modifications. Herein the bioconjugated oligonucleotides may contain the mono- or multivalent conjugation selected from moieties including, and not limited to, GalNAc, cholesterol, DL-tocopherol, saturated or unsaturated fatty acids such as palmitic acid, behenic acid, linoleic acid, and/or linolenic acid. Herein the bioconjugated oligonucleotides may contain the mono- or multivalent conjugation selected from moieties including, and not limited to, TFA amino, alkyne, fluorescein, biotin, pyrene, dabsyl and dabcyl moieties or their post synthetically modified moieties. Herein the bioconjugated oligonucleotides may contain moieties including, and not limited to, POM protected (E)-vinylphosphonate (EVP) or chemical phosphorylation reagent (CPR)-derived phosphate functional moieties. Herein the bioconjugated oligonucleotides may contain moieties including, and not limited to, nucleoside sugar modification, nucleosidic base modification, non-radioactive labels, nucleic acid cross-linking, and/or internucleotide linkage modification.

The present disclosure relates to the efficient cleavage and deprotection method for oligonucleotide from solid support. Herein the solid support is selected from supports including, and not limited to, Universal Support, Universal Support II, Universal Support III, UnyLinker™ (R=Phenyl or Isopropyl, AM Chemicals) and/or UnySupport™ (R=Methyl, Glen Research or ChemGenes), where the backbone structure is composed of 5,6-dihydroxy-2-alkyl (R=methyl, isopropyl, phenyl) hexahydro-1H-4,7-epoxyisoindole-1,3(2H)-dione. One of 5,6-dihydroxyl group is protected with trityl group such as TMT (trimethoxytrityl) or DMT (dimethoxytrityl) and the other is connected to the solid support through succinate linker. Additionally, UnyLinker and/or UnySupport can include the variants of the R group including alkyl groups such as isopropyl, and aryl groups such as toluenyl or benzyl.

The present disclosure relates to the efficient cleavage and deprotection method for oligonucleotide from solid support. Herein the cleavage and deprotection method may be the lithium assisted cleavage and deprotection method contributed to reducing the reaction time and showing a remarkable reduction of nucleophile-driven by-products.

Mirzabekov and colleagues (Nucleic Acids Research, 2000, vol. 28, No. 8, e29) showed that the procedure of rapid deprotection for synthetic oligodeoxynucleotides, utilizing the ammonia-free reagent mixture composed of triethylamine and lithium hydroxide in methanol, avoided additional purification steps. The representative condition was the cleavage and deprotection with a mixture of 0.5 M aqueous lithium hydroxide and 3.5 M triethylamine in methanol (1:10 v/v) at 75° C. for 60 min, followed by quenching with glacial acetic acid at −20° C. Kumar and colleagues (Nucleic Acids Research, 2002, vol. 30, No. 23, e130) revealed that the polyamine-assisted deprotection conditions was efficient to cleave the oligonucleotide chains from a cis-diol group bearing universal polymer support. They concluded that the use of spermine (1.0 M) in conjunction with lithium cations (0.5 M) in aqueous ammonia (32%) at 80° C. efficiently cleaved the oligonucleotides from the universal support. Zlatev and colleagues (Tetrahedron, 2018, 74, 6182-6186) developed a simple one-step cleavage and deprotection procedure with a 3% solution of diethylamine (DEA) in aqueous ammonia at either 35° C. for 20 h or 60° C. for 5 h for the 5′-POM protected (E)-vinylphosphonate conjugated oligonucleotide. Unfortunately, when we tested for several bioconjugated oligonucleotides with those suggested cleavage and deprotection methods, several unreported by-products were found, which could not be detected or identified with the technology at that time.

A new cleavage and deprotection method was developed first for tri-GalNAc bioconjugated oligonucleotides where the linear tri-GalNAc conjugation was implemented utilizing non-nucleosidic GalNAc phosphoramidite from AM Chemicals. An initial approach was pursued to find the source and concentration of metal cation in concentrated (28-30%) aqueous ammonia solution (1 mL/μmol) at ambient temperature (herein, temperature was fixed at 55° C.). The combination of 0.1 M, 1.0 M, 2.0 M, and 4.0 M concentration with various metal sources with chloride complex (LiCl, NaCl, KCl, RbCl, CsCl, BeCl₂, MgCl₂, CaCl₂, SrCl₂, BaCl₂, MnCl₂, FeCl₂, FeCl₃, CoCl₂, NiCl₂, CuCl, CuCl₂, ZnCl₂, and/or their hydrates) were tested for the cleavage and deprotection using tri-GalNAc bioconjugated oligonucleotides. Candidates for the metal sources were selected from solubility and color in aqueous ammonia solution, concentration-dependent cleavage and deprotection period, stability of oligonucleotide, commercial availability, and ease of handling the metal chloride complexes or their hydrates. As a result, the metal cation was selected from lithium chloride (Table 1A).

| SEQ ID | | FLP |
|---|---|---|
| NO | Sequence ID (5′ → 3′) | MW |
| SEQ ID | (mU)*(fG)*(mU)(fG)(fC)(fC)(mU)(mU) | 7023.1 |
| NO: 1 | (mC)(mU)(mC)(fA)(mU)(mC)(mU)(mA)(GalNAc)₃ | |

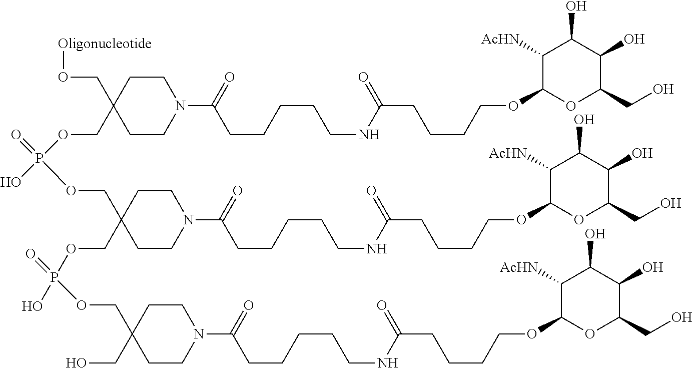

TABLE 1A
Selection of metal cation source in chloride complex for
tri-GalNAc bioconjugated oligonucleotide (SEQ ID NO: 1)

| Metal | Water | | Cleavage and |
|---|---|---|---|
| cation | solubility | | deprotection |
| source | (g/100 mL) | color | (C&D) Result |
| LiCl | 84 g, 25° C. | White solid | +++ |
| NaCl | 36 g, 25° C. | Colorless cubic | + |
| | | crystals | |
| KCl | 34 g, 20° C. | White crystalline | + |
| | | solid | |
| RbCl | 91 g, 20° C. | White crystals | + |
| CsCl | 191 g, 25° C. | White solid | + |
| BeCl₂ | 15 g, 20° C. | White to yellow | −− |
| | | crystals | |
| MgCl₂•6H₂O | 54 g, 20° C. | White crystalline | + |
| | | powder | |
| CaCl₂•2H₂O | 81 g, 25° C. | White solid | − |
| SrCl₂ | 53 g, 20° C. | White crystalline | − |
| | | powder | |
| BaCl₂•2H₂O | 35 g, 20° C. | White powder | − |
| MnCl₂•4H₂O | 73 g, 20° C. | Pink solid | + |
| FeCl₂•2H₂O | 68 g, 20° C. | Pale green powder | −− |
| FeCl₃•6H₂O | 91 g, 20° C. | Yellow solid | −− |
| CoCl₂•6H₂O | 52 g, 20° C. | Green crystals | −− |
| NiCl₂ | 67 g, 25° C. | White to off-white | + |
| | | powder | |
| CuCl | insoluble, | White powder | −−− |
| | 20° C. | | |
| CuCl₂•2H₂O | 75 g, 20° C. | Blue-green powder | − |
| ZnCl₂ | 432 g, 20° C. | White | + |

+++: strongly concentration-dependent cleavage and deprotection
++: weakly concentration-dependent cleavage and deprotection
+ or −: low or no concentration-dependent cleavage and deprotection
−− or −−−: solubility, color, and other issue (oligonucleotide decomposition) during cleavage and deprotection
Water solubility was collected from website (PubChem; https://pubchem.ncbi.nlm.nih.gov/).

Then, the purpose of the second approach was focused to find the proper lithium source at fixed 2.0 M concentration of lithium cation in concentrated (28-30%) aqueous ammonia solution (1.0 mL/μmol) from lithium fluoride (LiF), lithium chloride (LiCl), lithium bromide (LiBr), lithium iodide (LiI), lithium acetate (LiOAc), lithium tetraborate (Li₂B₄O₇), lithium hydroxide (LiOH), lithium carbonate (Li₂CO₃), lithium sulfate (Li₂SO₄), lithium nitrate (LiNO₃), and/or lithium perchlorate (LiClO₄), or their hydrates. LiCl, LiBr, LiI·3H₂O showed the faster cleavage and deprotection at 55° C. after 10-14 hours in one step. Lithium fluoride or lithium carbonate was not tested due to intrinsic low solubility in concentrated (28-30%) aqueous ammonia solution. Lithium hydroxide caused a lot of by-products during the cleavage and deprotection process. Lithium sulfate, lithium nitrate and lithium perchlorate also lead the concentration-dependent cleavage and deprotection, but took a longer time than lithium halides, causing more by-products (Table 2A).

| SEQ ID | | FLP |
|---|---|---|
| NO | Sequence ID (5′→3′) | MW |
| SEQ ID | (mU)*(fG)*(mU)(fG)(fC)(fC)(mU)(mU)(mC)(mU)(mC) | 7023.1 |
| NO: 1 | (fA)(mU)(mC)(mU)(mA)(GalNAc)₃ | |

TABLE 2A
Selection of lithium cation source from various salts for
tri-GalNAc bioconjugated oligonucleotide (SEQ ID NO: 1)

| Lithium | Water | | |
|---|---|---|---|
| cation | solubility | | C&D |
| source | (g/100 mL) | color | Result |
| LiF | insoluble, | White powder | − |
| | 25° C. | | |
| LiCl | 84 g, 25° C. | White solid | +++ |
| LiBr | 166 g, 25° C. | White solid | ++ |
| LiI•3H₂O | 167 g, 25° C. | White crystalline solid | ++ |
| LiOAc | 45 g, 25° C. | Whit crystalline solid | ++ |
| Li₂B₄O₇ | <10 g, 25° C. | White powder | + |
| LiOH•H₂O | 22 g, 10° C. | White solid | − |
| Li₂CO₃ | 1.3 g, 25° C. | White powder | − |
| Li₂SO₄•H₂O | 34 g, 25° C. | White crystalline solid | + |
| LiNO₃ | 52 g, 20° C. | White to light yellow | + |
| | | solid | |
| LiClO₄•3H₂O | 59 g, 25° C. | White crystals | + |

+++: completion of cleavage and deprotection within given period
++: completion of cleavage and deprotection within given period plus 4 hours
+: completion of cleavage and deprotection within given period plus 16 hours
−: solubility, color, and other issue (oligonucleotide decomposition) during cleavage and deprotection

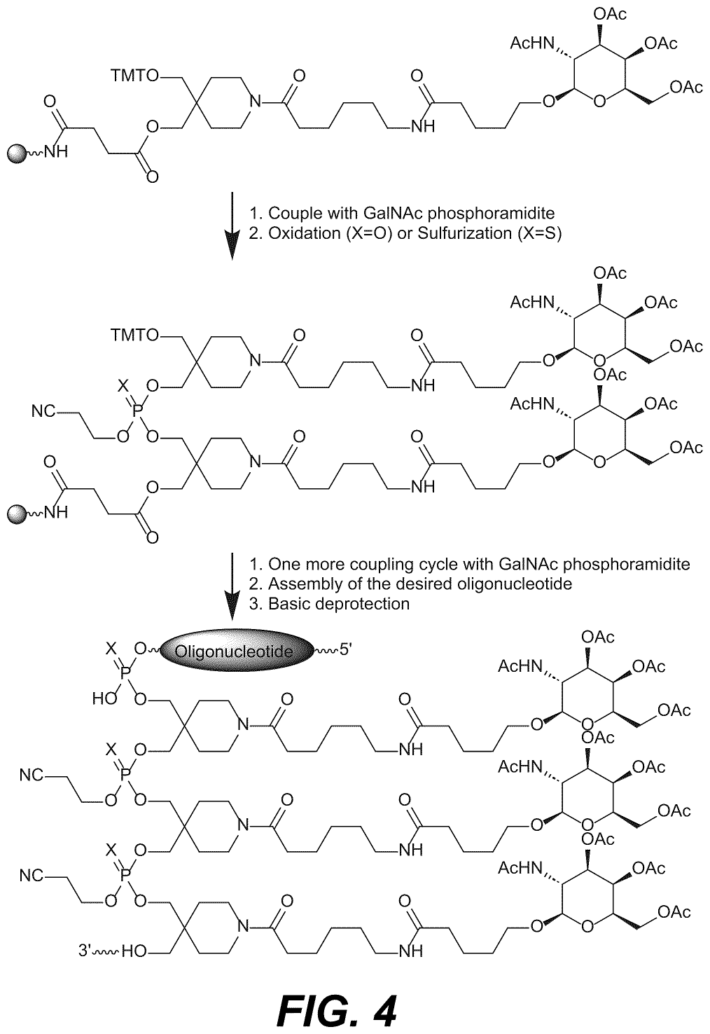

Next experiment was designed to determine the proper concentration, temperature, reaction time, and solution quantity for the efficient cleavage and deprotection for tri-GalNAc bioconjugated oligonucleotides with lithium chloride in concentrated (28-30%) aqueous ammonia solution in 1 μmol scale (Table 3A). Completion of cleavage and deprotection was determined by HT/LCMS, especially, to check the UnySupport tether and N-Ac protection groups. Compared to the previously reported two-step cleavage and deprotection method, the lithium assisted cleavage and deprotection method showed the lower impurity profiles. Those impurities were below the limit of quantification (LOQ) for PO impurity, FLP-GalNAc tether, FLP-GalNAc, and GalNAc anomer along with lower total impurities (FIG. 4). Interestingly, although cleavage was complete (UnySupport tethered oligonucleotide=0%), deprotection was incomplete to result in the N-Acetyl protection remaining under several conditions of too high concentration of lithium chloride over 5.0 M or 6.0 M, insufficient solution quantity, and/or low temperature treatment at 40° C. Thus, those combinations seemed to be important for the lithium assisted cleavage and deprotection.

| SEQ ID | | FLP |
|---|---|---|
| NO | Sequence ID (5′→3′) | MW |
| SEQ ID | (mU)*(fG)*(mU)(fG)(fC)(fC)(mU)(mU)(mC)(mU)(mC)(fA) | 7023.1 |
| NO: 1 | (mU)(mC)(mU)(mA)(GalNAc)₃ | |

TABLE 3A
Selection of optimal cleavage and deprotection condition with lithium
chloride for tri-GalNAc bioconjugated oligonucleotide (SEQ ID NO: 1)

| | | Reaction | | | UnySupport | |
|---|---|---|---|---|---|---|
| Concentration | Temperature | time | | Volume | tethered FLP | Deprotection |
| 1.0M (4.2%) | 40° C. | 16 | hours | 250 μL/μmol | 64% | N—Ac 0.9% |
| | 40° C. | 16 | hours | 500 μL/μmol | 53% | N—Ac 0.4% |
| | 40° C. | 16 | hours | 750 μL/μmol | 48% | Complete |
| | 55° C. | 16 | hours | 250 μL/μmol | 43% | Complete |
| | 55° C. | 16 | hours | 500 μL/μmol | 25% | Complete |
| | 55° C. | 16 | hours | 750 μL/μmol | 16% | Complete |
| 3.0M (12.6%) | 40° C. | 16 | hours | 250 μL/μmol | 42% | Complete |
| | 40° C. | 16 | hours | 500 μL/μmol | 14% | Complete |
| | 40° C. | 16 | hours | 750 μL/μmol | 4% | Complete |
| | 55° C. | 16 | hours | 100 μL/μmol | 2% | Complete |
| | 55° C. | 16 | hours | 250 μL/μmol | 2% | Complete |
| | 55° C. | 16 | hours | 500 μL/μmol | 1% | Complete |
| | 55° C. | 16 | hours | 750 μL/μmol | 0% | Complete |
| 4.0M (16.8%) | 40° C. | 24 | hours | 100 μL/μmol | 0% | N—Ac 1.7% |
| | 40° C. | 24 | hours | 250 μL/μmol | 0% | Complete |
| | 40° C. | 24 | hours | 500 μL/μmol | 0% | Complete |
| | 55° C. | 16 | hours | 100 μL/μmol | 0% | Complete |
| | 55° C. | 8 | hours | 250 μL/μmol | 0% | Complete |
| | 55° C. | 8 | hours | 500 μL/μmol | 0% | Complete |
| 5.0M (21.0%) | 55° C. | 8 | hours | 100 μL/μmol | 0% | N—Ac 1.7% |
| | 55° C. | 8 | hours | 250 μL/μmol | 0% | N—Ac 0.9% |
| | 55° C. | 8 | hours | 500 μL/μmol | 0% | Complete |
| 6.0M (25.2%) | 55° C. | 8 | hours | 100 μL/μmol | 0% | N—Ac 10.2% |
| | 55° C. | 8 | hours | 250 μL/μmol | 0% | N—Ac 4.2% |
| | 55° C. | 8 | hours | 500 μL/μmol | 0% | N—Ac 1.7% |

The lithium assisted cleavage and deprotection was also tested for several tri-GalNAc bioconjugated oligonucleotides in large scale from 10 μmol to 320 μmol with lithium chloride (4.0 M) in concentrated (28-30%) aqueous ammonia solution (100˜500 μL/μmol) at 55° C. for the given period (10 to 14 hours), resulting in completion of cleavage and deprotection along with low impurity profile (Table 4A).

| SEQ ID | | FLP |
|---|---|---|
| NO | Sequence ID (5′→3′) | MW |
| SEQ ID | (mU)*(fG)*(mU)(fG)(fC)(fC)(mU)(mU)(mC)(mU)(mC) | 7023.1 |
| NO: 1 | (fA)(mU)(mC)(mU)(mA)(GalNAc)₃ | |
| SEQ ID | (mG)*(mG)*(mA)(fC)(mA)(fG)(mA)(mG)(mU)(mU) | 7271.4 |
| NO: 2 | (mA)(mU)(mC)(mG)(mA)(mA)(GalNAc)₃ | |
| SEQ ID | (mG)*(fG)*(mA)(fC)(fA)(fG)(mA)(mG)(mU)(mU) | 7223.3 |
| NO: 3 | (mA)(mU)(mC)(fG)(mA)(fA)(GalNAc)₃ | |
| SEQ ID | (mG)*(fG)*(mC)(fU)(fC)(fC)(mU)(mU)(mC)(mU) | 7096.2 |
| NO: 4 | (mG)(mA)(mA)(fC)(mA)(fA)(GalNAc)₃ | |
| SEQ ID | (mG)*(fG)*(mC)(fU)(fC)(fC)(mU)(mU)(mC) | 7108.2 |
| NO: 5 | (mU)(mG)(fA)(mA)(mC)(mA)(mA)(GalNAc)₃ | |
| SEQ ID | (mA)*(fG)*(mA)(fA)(fC)(fU)(mU)(mG)(mC) | 7143.3 |
| NO: 6 | (mC)(mA)(mA)(mG)(fC)(mU)(fA)(GalNAc)₃ | |

TABLE 4A
Scale-up results of lithium assisted cleavage and deprotection

| Concentration | Sequence ID | Scale | Time | Volume | Cleavage | Deprotection |
|---|---|---|---|---|---|---|
| 4.0M | SEQ ID | 10 | 10 | 100 | complete | complete |
| (16.8%) | NO: 1 | μmol | hours | μL/μmol | | |
| | | 60 | 10 | 100 | complete | complete |
| | | μmol | hours | μL/μmol | | |
| | | 160 | 12 | 100 | complete | complete |
| | | μmol | hours | μL/μmol | | |
| | | 320 | 14 | 100 | complete | complete |
| | | μmol | hours | μL/μmol | | |
| | | 320 | 11 | 200 | complete | complete |
| | | μmol | hours | μL/μmol | | |
| | | 320 | 9 | 500 | complete | complete |
| | | μmol | hours | μL/μmol | | |
| | SEQ ID | 160 | 10 | 500 | complete | complete |
| | NO: 2 | μmol | hours | μL/μmol | | |
| | SEQ ID | 160 | 10 | 500 | complete | complete |
| | NO: 3 | μmol | hours | μL/μmol | | |
| | SEQ ID | 160 | 10 | 500 | complete | complete |
| | NO: 4 | μmol | hours | μL/μmol | | |
| | SEQ ID | 160 | 10 | 500 | complete | complete |
| | NO: 5 | μmol | hours | μL/μmol | | |
| | SEQ ID | 160 | 10 | 500 | complete | complete |
| | NO: 6 | μmol | hours | μL/μmol | | |

Thus, the present disclosure relates to the efficient cleavage and deprotection method for tri-GalNAc containing bioconjugated oligonucleotides from solid support. Herein the cleavage and deprotection method may contain the lithium cation provided from various lithium sources such as lithium fluoride, lithium chloride, lithium bromide, and lithium iodide, lithium acetate, lithium tetraborate, lithium hydroxide, lithium carbonate, lithium sulfate, lithium nitrate, and/or lithium perchlorate, or their hydrate form. In some embodiments, the lithium source is a lithium chloride, a lithium bromide, or a lithium iodide. In some embodiments, the lithium source is a lithium chloride. Concentration of lithium cation can be selected from 0.1 M to 6.0 M, which are corresponding to 0.1% to 40% (w/v) depending on the molecular weight of lithium sources. In some embodiments, the concentration of lithium cation is 3.0 M to 5.0 M. In some embodiments, the concentration of lithium cation is 3.5 M to 4.5 M. In some embodiments, the concentration of lithium cation is 4.0 M. Temperature can be selected from RT to 80° C. In some embodiments, the temperature is 30° C. to 70° C. In some embodiments, the temperature is 40° C. to 55° C. Reaction time can be selected from 4 hours to 48 hours. In some embodiments, the reaction time is 6 hours to 24 hours. In some embodiments, the reaction time is 8 hours to 14 hours. Solution quantity can be selected from 100 μL/μmol to 1.0 mL/μmol. In some embodiments, the solution quantity can be 100 μL/μmol to 500 μL/μmol. In some embodiments, the solution quantity can be 100 μL/μmol to 250 μL/μmol. During the cleavage and deprotection, the container can be located in heat block without shaking or rocking or can be shaken or rocked at heating incubator with the given temperature (usually, shaking or rocking may accelerate the cleavage and deprotection, resulting in less impurities).

Then, the lithium assisted cleavage and deprotection method was tested for several bioconjugated oligonucleotides containing cholesterol, DL-tocopherol, saturated or unsaturated fatty acids such as palmitic acid, behenic acid, linoleic acid, and/or linolenic acid, which were derived from AM Chemicals (Table 5A). General condition was as follows: 4 M Lithium chloride in concentrated (28-30%) aqueous ammonia solution (200 to 500 μL/μmol) at 40° C. for the bioconjugated oligonucleotides with unsaturated fatty acids (linoleic acid and linolenic acid) and at 55° C. for the other bioconjugated oligonucleotides with cholesterol, DL-tocopherol, and saturated fatty acid (palmitic acid). Reaction time was measured until the cleavage and deprotection was complete in 10 μmol scale.

Table 5A. Application of the lithium assisted cleavage and deprotection to various bioconjugated oligonucleotides containing non-nucleosidic modifier

The oligonucleotide used is SEQ ID NO: 7

5′(mU)*(mU)*(fA)(mA)(fU)(fC)(fC)(mU)(mC)(mA)(mC)
(mU)(mC)(mU)(mA)(mA)(mA)*(BioConjugation)

| | Temper- | | | | |
|---|---|---|---|---|---|
| Bioconjugation | ature | Time | Volume | Cleavage | Deprotection |
| Cholesterol | 55° C. | 10 | 200 | complete | complete |
| | | hours | μL/umol | | |

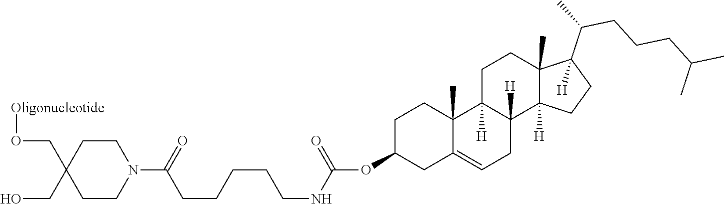

| | | | | | |
|---|---|---|---|---|---|
| DL-tocopherol | 55° C. | 10 | 200 | complete | complete |
| | | hours | μL/umol | | |

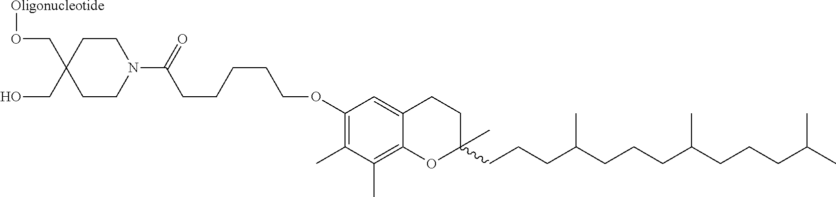

| | | | | | |
|---|---|---|---|---|---|
| Palmitic acid | 55° C. | 10 | 200 | complete | complete |
| | | hours | μL/umol | | |

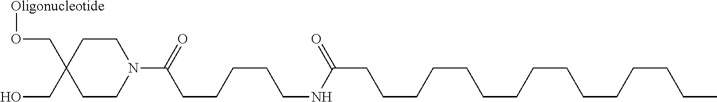

| | | | | | |
|---|---|---|---|---|---|
| Linoleic acid | 40° C. | 12 | 500 | complete | Complete |
| | | hours | μL/umol | | |

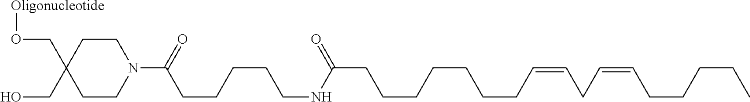

| | | | | | |
|---|---|---|---|---|---|
| Linolenic acid | 40° C. | 12 | 500 | complete | complete |
| | | hours | μL/umol | | |

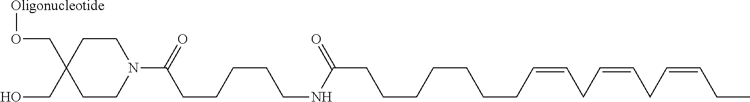

The lithium assisted cleavage and deprotection method was tested for oligonucleotides including and not limited to 5′ to 3′ reversely synthesized oligonucleotides containing fatty acid bioconjugation. Oligonucleotide was synthesized on solid support utilizing the RNA reverse amidite and Fmoc or ivDde protected Amino modifier C6 amidite, which was implemented for the post-synthetic modification with mono- or multivalent fatty acid conjugation. Bioconjugated oligonucleotide was treated with the lithium assisted cleavage and deprotection solution as follows, resulting in the completion of cleavage and deprotection at 55° C. within 14 hours with lower impurity profile, especially no PO impurities (Table 6A).

Table 6A. Application of the lithium assisted cleavage and deprotection for various reversely synthesized bioconjugated oligonucleotides containing post-synthetic modification with fatty acids

The oligonucleotide used is SEQ ID NO: 7

5′(mU)*(mU)*(fA)(mA)(fU)(fC)(fC)(mU)(mC)(mA)(mC)
(mU)(mC)(mU)(mA)(mA)(mA)*(BioConjugation)

| Bioconjugation | Temperature | Time | Volume | Cleavage | Deprotection |
|---|---|---|---|---|---|
| PA-(AmC6)- | 55° C. | 8 hours | 500 | complete | complete |
| 3′ | | | μL/umol | | |

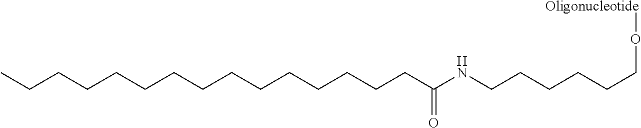

| | | | | | |
|---|---|---|---|---|---|
| PA-AEEA- | 55° C. | 8 hours | 500 | complete | complete |
| (AmC6)-3′ | | | μL/umol | | |

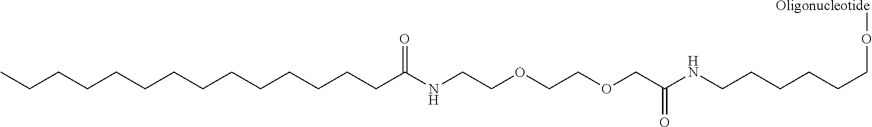

| | | | | | |
|---|---|---|---|---|---|
| BA-(AmC6)- | 55° C. | 8 hours | 500 | complete | complete |
| 3′ | | | μL/umol | | |

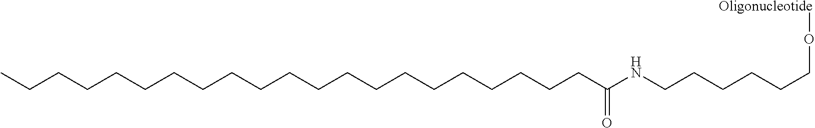

| | | | | | |
|---|---|---|---|---|---|
| BA-AEEA- | 55° C. | 8 hours | 500 | complete | complete |
| (AmC6)-3′ | | | μL/umol | | |

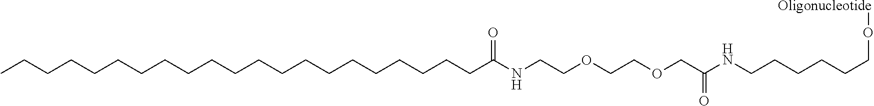

| | | | | | |
|---|---|---|---|---|---|
| (PA-AEEA)2- | 55° C. | 10 | 500 | complete | complete |
| (GABA-D- | | hours | μL/umol | | |
| Lys)- | | | | | |
| (AmC6)-3′ | | | | | |

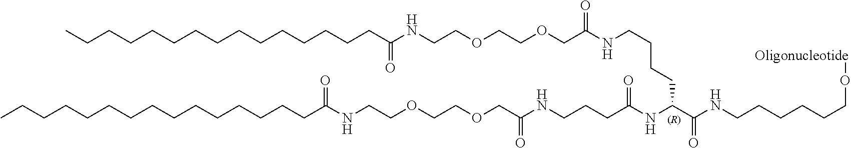

| | | | | | |
|---|---|---|---|---|---|
| (PA-AEEA)2- | 55° C. | 10 | 500 | complete | complete |
| (GABA-BH- | | hours | μL/umol | | |
| Lys)- | | | | | |
| (AmC6)-3′ | | | | | |

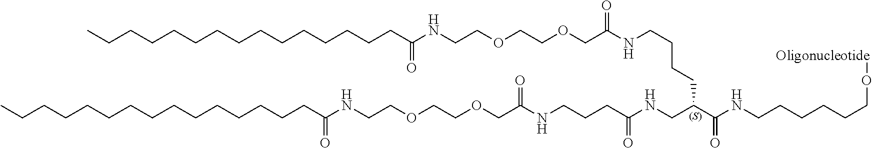

| | | | | | |
|---|---|---|---|---|---|
| PA/DDA- | 55° C. | 10 | 500 | complete | complete |
| (GABA-D- | | hours | μL/umol | | |
| Lys)- | | | | | |
| (AmC6)-3′ | | | | | |

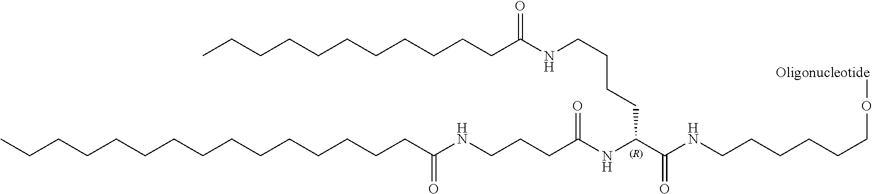

| | | | | | |
|---|---|---|---|---|---|
| PA/TDA- | 55° C. | 10 | 500 | complete | complete |
| (GABA-D- | | hours | μL/umol | | |
| Lys)- | | | | | |
| (AmC6)-3′ | | | | | |

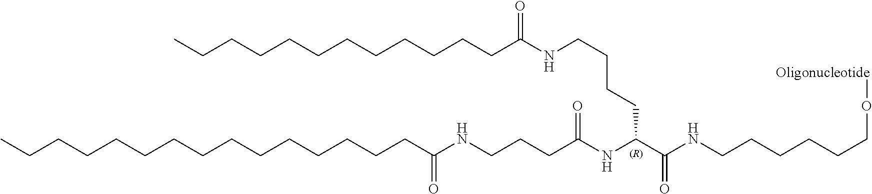

| | | | | | |
|---|---|---|---|---|---|
| PA/MA- | 55° C. | 10 | 500 | complete | complete |
| (GABA-D- | | hours | uL/umol | | |
| Lys)- | | | | | |
| (AmC6)-3′ | | | | | |

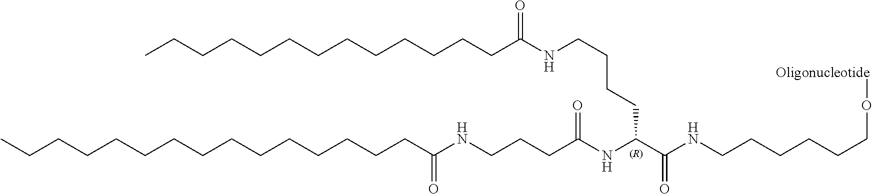

| | | | | | |
|---|---|---|---|---|---|
| PA/PDA- | 55° C. | 10 | 500 | complete | complete |
| (GABA-D- | | hours | μL/umol | | |
| Lys)- | | | | | |
| (AmC6)-3′ | | | | | |

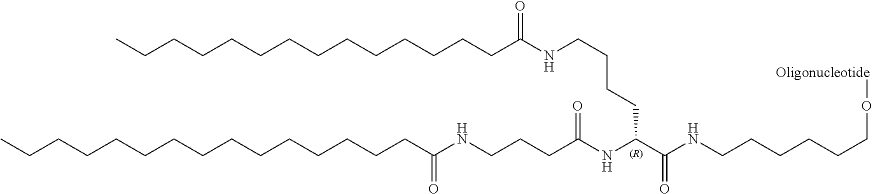

| | | | | | |
|---|---|---|---|---|---|
| (GalNAc)3- | 55° C. | 14 | 500 | complete | complete |
| (GABA-D- | | hours | μL/umol | | |
| Lys-BH-Lys)- | | | | | |
| (AmC6)-3′ | | | | | |

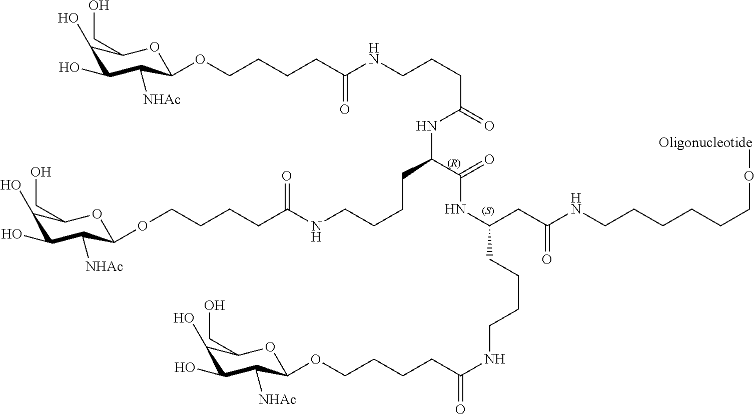

PA: Palmitic acid/BA: Behenic acid
AEEA: Aminoethoxyethoxyacetic acid/GABA: gamma-aminobutyric acid
DDA: Dodecanoic acid (C12)
TDA: Tridecanoic acid (C13)
MA: Myristic acid or tetradecanoic acid (C14)
PDA: Pentadecanoic acid (C15)
D-Lys: D-Lysine/BH-Lys: Beta-Homolysine

Later, without being bound by theory, it was considered why cleavage and deprotection for those bioconjugated oligonucleotides were not easily complete with the general cleavage and deprotection methods. There was found that those bioconjugated oligonucleotides were connected to the solid support through primary alcoholic phosphate linkage. There was the phosphate linkage between solid support and primary alcohol of piperidinyl conjugation linker for bioconjugated oligonucleotides containing mono- or multivalent GalNAc, cholesterol, DL-tocopherol, saturated or unsaturated fatty acids such as palmitic acid, behenic acid, linoleic acid, and/or linolenic acid (See Scheme 2). There was the phosphate linkage between solid support and 5′-primary alcohol of nucleoside for reversely (5′ to 3′) synthesized bioconjugated oligonucleotides containing (later, post-synthetically modified with) mono- or multivalent fatty acid conjugation (See Scheme 3). During the cleavage and deprotection, solid support and succinate moiety was considered to be cleaved at first, resulting in the UnySupport tether attached to primary alcohol of bioconjugated oligonucleotide through the phosphate linkage. One secondary alcohol from UnySupport tether would be negatively charged, and another oxygen adjacent to phosphorus at phosphate would be negatively charged as well, resulting in strong repulsion between two negatively charged oxygens. Otherwise, one secondary alcohol from UnySupport tether would attack to phosphorus to form the cyclophosphate to kick out the oligonucleotide, which was the completion of cleavage. Without being theory bound, lithium was believed to coordinate as cation on those alcohol and/or oxygen, resulting in decreasing the anionic repulsion and completion of cleavage (Scheme 4 and Scheme 5).

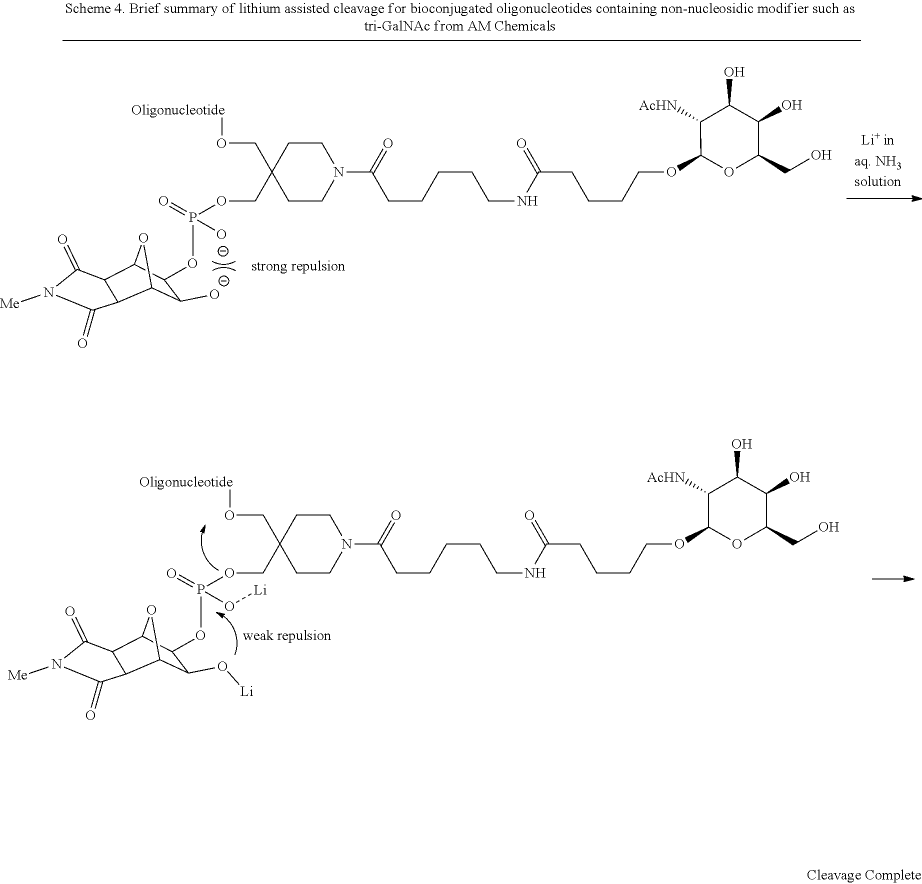

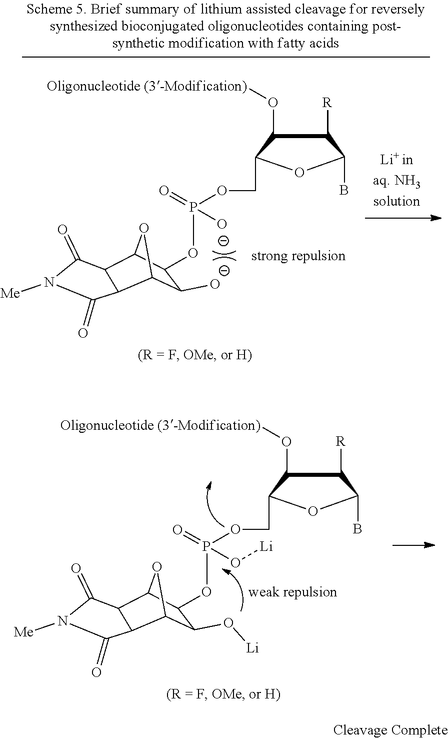

The lithium assisted cleavage and deprotection method was also tested for 5′-bioconjugated oligonucleotides containing the POM protected (E)-vinylphosphonate (EVP) or phosphate (originally, from Solid CPR II, as DMT-On). For the 5′-POM protected EVP conjugated oligonucleotide, the lithium assisted cleavage and deprotection showed the improved impurity profile with no PO impurity. However, there were observed the acrylonitrile migration impurity and FLP-52 m/z impurity (removal of acrylaldehyde from uracil) under the given condition, when treated with 4.0 M lithium chloride in concentrated (28-30%) aqueous ammonia solution (100 μL/μmol) at 55° C. for 14 hours. Although previously reported that 3% diethylamine addition showed the completion of cleavage and deprotection, it was not possible to avoid the diethylamine adduct or PO impurities. To decrease or remove those impurities, various alkylamines were tested as additive to the current lithium assisted cleavage and deprotection method and those progress and two impurities were monitored by HT/LCMS (Table 7A).

| SEQ ID | | FLP |
|---|---|---|
| NO | Sequence ID (5′ → 3′) | MW |
| SEQ ID | (EVP)(mU)*(fU)*(mU)(mA)(mG)(fA)(mG)(fU)(fG)(mA) | 7883.2 |
| NO: 8 | (mG)(mG)(mA)(fU)(mU)(fA)(mA)(mA) | |
| | (mA)*(mU)*(mG)*(mA)*(mG) | |

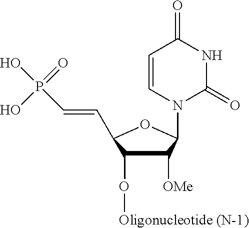

TABLE 7A
The lithium assisted cleavage and deprotection for the POM protected (E)-vinylphosphonate bioconjugated
oligonucleotides (Seq. ID.: 23) with alkylamine additives at 55° C. for 14 hours

| | Alkyl | | | | | | | |
|---|---|---|---|---|---|---|---|---|
| | amine | | | Cleavage and | | | Acrylonitrile | Acrylaldehyde |
| Alkyl | Amount | | | deprotection | PO | Amine | migration | removal |
| amines | (v/v) | Conc | Volume | results | impurity | adduct | adduct | adduct |
| Methyl | 20% | N/A | 100 | Complete | 0% | 10.1% | 0% | 0% |
| amine | | | μL/μmol | | | | | |
| | | 1.0M | 100 | Complete | 0% | 5.0% | 0% | 0% |
| | | | μL/μmol | | | | | |
| | | 2.0M | 100 | Complete | 0% | 4.3% | 0% | 0% |
| | | | μL/μmol | | | | | |
| | | 4.0M | 100 | Complete* | 0% | 0% | 0% | 0% |
| | | | μL/μmol | | | | | |
| | | 6.0M | 100 | Complete* | 0% | 0% | 0% | 0% |
| | | | μL/μmol | | | | | |
| Methyl | 50% | N/A | 100 | Complete | 0% | 13.9% | 0% | 1.4% |
| amine | | | μL/μmol | | | | | |
| | | 1.0M | 100 | Complete | 0% | 7.2% | 0% | 0% |
| | | | μL/μmol | | | | | |
| | | 2.0M | 100 | Complete | 0% | 5.3% | 0% | 0% |
| | | | μL/μmol | | | | | |
| | | 4.0M | 100 | Complete* | 0% | 0% | 0% | 0% |
| | | | μL/umol | | | | | |
| | | 6.0M | 100 | Complete* | 0% | 0% | 0% | 0% |
| | | | μL/μmol | | | | | |
| Diethyl | 3% | N/A | 100 | incomplete | 3.4% | 1.7% | 2.2% | 1.7% |
| amine | | | μL/μmol | (N—Ac 1.2%) | | | | |
| | | 1.0M | 100 | Complete | 0% | 1.6% | 1.4% | 1.7% |
| | | | μL/μmol | | | | | |
| | | 2.0M | 100 | Complete | 0% | 0.9% | 1.4% | 1.7% |
| | | | μL/μmol | | | | | |
| | | 4.0M | 100 | Complete | 0% | 0.9% | 0.9% | 1.9% |
| | | | μL/μmol | | | | | |
| | | 6.0M | 100 | Complete* | 0% | 0.4% | 0.6% | 1.8% |
| | | | μL/μmol | | | | | |
| Triethyl | 3% | N/A | 100 | incomplete, | 1.6% | 0% | 3.6% | 3.2% |
| amine | | | μL/μmol | N—Ac 2.0% | | | | |
| | | 1.0M | 100 | incomplete, | 0% | 0% | 2.7% | 2.5% |
| | | | μL/μmol | N—Ac 1.5% | | | | |
| | | 2.0M | 100 | incomplete, | 0% | 0% | 1.8% | 1.7% |
| | | | μL/μmol | N—Ac 1.0%) | | | | |
| | | 4.0M | 100 | incomplete, | 0% | 0% | 1.6% | 0% |
| | | | μL/μmol | N—Ac 1.1% | | | | |
| | | 6.0M | 100 | incomplete, | 0% | 0% | 1.9% | 0% |
| | | | μL/μmol | N—Ac 0.8% | | | | |
| Triethyl | 5% | 2.0M | 100 | 6 h, | 0% | 0% | 1.8% | 0.9% |
| amine | | | μL/μmol | incomplete, | | | | |
| | | | | N—Ac 1.7% | | | | |
| | | 2.0M | 100 | 14 h, | 0% | 0% | 2.2% | 1.6% |
| | | | μL/μmol | incomplete, | | | | |
| | | | | N—Ac 1.0% | | | | |
| Triethyl | 10% | 2.0M | 100 | 6 h | 0% | 0% | 0% | 0% |
| amine | | | μL/μmol | incomplete**, | | | | |
| | | | | N—Ac 1.4% | | | | |
| | | 2.0M | 100 | 14 h, | 0% | 0% | 0% | 0% |
| | | | μL/μmol | complete** | | | | |
| Di- | 3% | N/A | 100 | Complete | 3.3% | 0% | 3.1% | 2.8% |
| isopropyl | | | μL/μmol | | | | | |
| ethyl | | 1.0M | 100 | Complete | 1.6% | 0% | 3.8% | 1.7% |
| amine | | | μL/μmol | | | | | |
| | | 2.0M | 100 | Complete | 1.3% | 0% | 2.4% | 1.6% |
| | | | μL/μmol | | | | | |
| | | 4.0M | 100 | Complete* | 0% | 0% | 0% | 0% |
| | | | μL/μmol | | | | | |
| | | 6.0M | 100 | Complete* | 0% | 0% | 0% | 0% |
| | | | μL/μmol | | | | | |

*Total quantity of bioconjugated oligonucleotide decreased due to partial degradation
**For 10% TEA, cleavage and deprotection was complete after 14 hours without increasing impurities.
*** Impurities observed during the cleavage and deprotection

| | | MW change from |
|---|---|---|
| Impurities | Representative structure | FLP |
| Amine adduct | 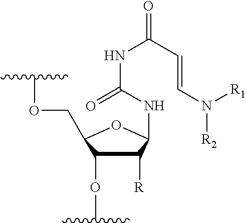 | Methylamine, +31 Da Diethylamine, +73 Da |
| Acrylonitrile migration adduct | 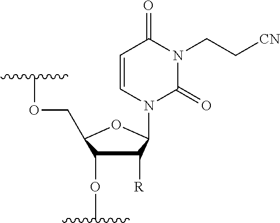 | Acrylonitrile, +53 Da |
| Acrylaldehyde removal adduct | 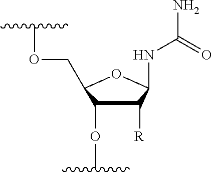 | Acrylaldehyde, −52 Da |

Because the lithium assisted cleavage and deprotection with alkylamine showed the improved results when using the 10% triethylamine as additive, the following experiments were conducted for 5′-phosphate containing oligonucleotides (originally, from Solid CPR II). General condition was as follows: 2.0 M lithium chloride, 10% triethylamine in concentrated (28-30%) aqueous ammonia solution (100-500 μL/μmol) at 40-55° C. for 8-14 hours (sometimes the treatment at high temperature for long time lead the partial degradation of oligonucleotides, so temperature was adjusted to 40° C. for 5′-phosphate conjugated oligonucleotides (Table 8A).

| SEQ ID | | FLP |
|---|---|---|
| NO | Sequence ID (5′ → 3′) | MW |
| SEQ ID | (Phos)(mU)*(fU)*(mU)(mA)(mG)(fA)(mG)(fU)(fG)(mA) | 7887.2 |
| NO: 9 | (mG)(mG)(mA)(fU)(mU)(fA)(mA)(mA)(mA)*(mU)* | |
| | (mG)*(mA)*(mG) | |
| SEQ ID | (Phos)(mU)*(fA)*(mA)(mG)(mA)(mG)(fA)(mA)(fA)(mA) | 6538.4 |
| NO: 10 | (mC)(mU)(mG)(fG)(mU)*(fC)*(mA)*(mG)*(mA) | |
| SEQ ID | (Phos)(mU)*(fA)*(mU)(mC)(mA)(mG)(fG)(mA)(fG)(mU) | 6492.3 |
| NO: 11 | (mG)(mA)(mC)(fU)(mG)*(fA)*(mA)*(mA)*(mU) | |

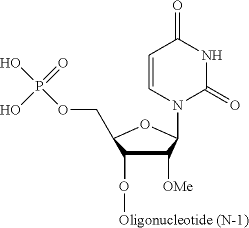

TABLE 8A
Lithium assisted cleavage and deprotection for the 5′-phosphate bioconjugated
oligonucleotides with 10% triethylamine additive at 40-55° C. for 8-18 hours

| SEQ ID NO | Bioconjugation | Temperature | Time | Solution | Cleavage | Deprotection |
|---|---|---|---|---|---|---|
| SEQ ID NO: | Solid CPR II | 40° C. | 20 | 100 | complete | complete |
| 9 | (5′-Phosphate) | | hours | μL/μmol | | |
| SEQ ID NO: | Solid CPR II | 40° C. | 14 | 500 | complete | complete |
| 9 | (5′-Phosphate) | | hours | μL/μmol | | |
| SEQ ID NO: | Solid CPR II | 55° C. | 17 | 100 | Complete* | complete |
| 9 | (5′-Phosphate) | | hours | μL/μmol | | |
| SEQ ID NO: | Solid CPR II | 55° C. | 10 | 500 | Complete | complete |
| 9 | (5′-Phosphate) | | hours | μL/μmol | | |
| SEQ ID NO: | Solid CPR II | 40° C. | 14 | 500 | complete | complete |
| 10 | (5′-Phosphate) | | hours | μL/μmol | | |
| SEQ ID NO: | Solid CPR II | 40° C. | 14 | 500 | complete | complete |
| 11 | (5′-Phosphate) | | hours | μL/μmol | | |

*5′-Phosphate conjugated oligonucleotide was partially decomposed

Additionally, the stability (retention of efficacy) of the lithiated aqueous ammonium hydroxide solution was verified by using the stored reagent periodically over a period of time to synthesize SEQ ID NO:12. To conduct the stability test, a reagent-solution comprising 2.0 M lithium chloride and 10% triethylamine in concentrated (28%-30%) aqueous ammonia solution was prepared and stored in a refrigerator (4° C.) and at room temperature (15-25° C.). The stored reagent-solution was thoroughly mixed each time and used for cleavage and deprotection.

General condition for cleavage and deprotection was as follows: 2.0 M lithium chloride, 10% triethylamine in concentrated (28%-30%) aqueous ammonia solution (250 μL/μmol) at 50° C. for 14 hours using 2 μmol of oligonucleotide. The stored reagent-solution was used over a period of 24 months (Table 10A, Table 11A). The retention of efficacy was analyzed by monitoring the purity of oligonucleotide following cleavage and deprotection of the POM protected (E)-vinylphosphonate (EVP) bioconjugated oligonucleotides (SEQ. ID NO: 12).

| SEQ | | |
|---|---|---|
| ID | | FLP |
| NO | Sequence ID (5′ → 3′) | MW |
| SEQ | (EVP)(mU) *(fA)*(mA)(mA)(mG)(fG)(mU)(mG)(mC) | 6810.65 |
| ID | (mA)(mA)(mA)(mC)(fA)(mU)(fG)(mU)*(mA)* | |
| NO: | (mA)*(mC) | |
| 12 | | |

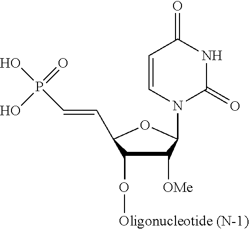

TABLE 10A
Testing of lithiated aqueous ammonium hydroxide solution stored at 4° C.

| | | C&D | | | | |
|---|---|---|---|---|---|---|
| | | Solution | | | | |
| SEQ ID NO | Bioconjugation | Volume | Period* | | Purity** | Total OD*** |
| SEQ ID NO: 12 | EVP | 250 μL/μmol | Initial | | 93.83% ± 0.83% | 283.67 ± 1.92 |
| SEQ ID NO: 12 | EVP | 250 μL/μmol | 4 | days | 94.47% ± 0.16% | 277.80 ± 2.34 |
| SEQ ID NO: 12 | EVP | 250 μL/μmol | 7 | days | 94.86% ± 0.28% | 285.24 ± 2.55 |
| SEQ ID NO: 12 | EVP | 250 μL/μmol | 11 | days | 92.09% ± 0.59% | 284.24 ± 1.91 |
| SEQ ID NO: 12 | EVP | 250 μL/μmol | 14 | days | 92.87% ± 1.45% | 285.14 ± 0.12 |
| SEQ ID NO: 12 | EVP | 250 μL/μmol | 21 | days | 92.05% ± 2.93% | 283.44 ± 3.12 |
| SEQ ID NO: 12 | EVP | 250 μL/μmol | 1 | M | 95.24% ± 0.17% | 284.84 ± 2.29 |
| SEQ ID NO: 12 | EVP | 250 μL/μmol | 2 | M | 94.60% ± 0.11% | 282.57 ± 0.56 |
| SEQ ID NO: 12 | EVP | 250 μL/μmol | 3 | M | 94.52% ± 1.36% | 282.60 ± 0.46 |
| SEQ ID NO: 12 | EVP | 250 μL/μmol | 6 | M | 94.06% ± 1.73% | 279.57 ± 0.48 |
| SEQ ID NO: 12 | EVP | 250 μL/μmol | 9 | M | 95.01% ± 0.32% | 282.64 ± 0.08 |
| SEQ ID NO: 12 | EVP | 250 μL/μmol | 12 | M | 95.16% ± 0.63% | 281.57 ± 0.76 |

TABLE 11A
Testing of lithiated aqueous ammonium hydroxide solution stored at room temperature

| | | C&D | | | | |
|---|---|---|---|---|---|---|
| | | Solution | | | | |
| SEQ ID NO | Bioconjugation | Volume | Period* | | Purity** | Total OD*** |
| SEQ ID NO: 12 | EVP | 250 μL/μmol | Initial | | 93.83% ± 0.83% | 283.67 ± 1.92 |
| SEQ ID NO: 12 | EVP | 250 μL/μmol | 2 | days | 94.47% ± 1.28% | 283.87 ± 0.84 |
| SEQ ID NO: 12 | EVP | 250 μL/μmol | 9 | days | 94.86% ± 1.43% | 286.00 ± 1.87 |
| SEQ ID NO: 12 | EVP | 250 μL/μmol | 16 | days | 92.09% ± 0.61% | 276.04 ± 2.09 |
| SEQ ID NO: 12 | EVP | 250 μL/μmol | 1 | M | 92.87% ± 0.77% | 283.14 ± 0.31 |
| SEQ ID NO: 12 | EVP | 250 μL/μmol | 2 | M | 92.05% ± 0.33% | 281.74 ± 2.19 |
| SEQ ID NO: 12 | EVP | 250 μL/μmol | 3 | M | 95.24% ± 1.45% | 277.27 ± 5.78 |
| SEQ ID NO: 12 | EVP | 250 μL/μmol | 6 | M | 94.60% ± 0.31% | 275.70 ± 0.20 |
| SEQ ID NO: 12 | EVP | 250 μL/μmol | 9 | M | 94.52% ± 1.35% | 281.20 ± 1.38 |
| SEQ ID NO: 12 | EVP | 250 μL/μmol | 12 | M | 94.06% ± 1.28% | 282.80 ± 1.84 |

*Period meant the elapsed time from the date of solution preparation.
**Purity was monitored by HTCS (High-throughput characterization system by Novatia).
***The measurement of total OD (optical density) was conducted through UV spectrophotometry.

Provided herein is a composition, which is an aqueous composition comprising about 0.1 to about 40% w/w of one or more of a lithium salt, and wherein the composition is at least about 50% saturated with ammonia. As used herein, a saturated solution of ammonia comprises about 35.6% w/w of ammonia, or about 308 grams of ammonia per litre of solution, e.g., a solution at about 15-16° C. A composition at least about 50% saturated with ammonia comprises at least about 18% w/w of ammonia. In some embodiments, an aqueous composition described herein comprises about 15% to about 30% w/w of ammonia. In some embodiments, an aqueous composition described herein comprises about 18% to about 32% w/w of ammonia. In some embodiments, an aqueous composition described herein comprises about 20% to about 30% w/w of ammonia. In some embodiments, an aqueous composition described herein comprises about 25% to about 30% w/w of ammonia. In some embodiments, an aqueous composition described herein comprises about 15% to about 20% w/w of ammonia. In some embodiments, the concentrations of ammonia described refer to solutions having a temperature of about-5 to about 55° C., e.g., about 0, 4, 10, 15, 20, 25, or even up to about 50 or 55° C.

In some embodiments, the composition further comprises one or more of an alkylamine, wherein the one or more alkylamine is present at about 1% to about 40% w/w.

In some embodiments, the one or more lithium salt includes a lithium cation and one or more of an anion, wherein the one or more anion has, independently, a molar mass of at least 18 g/mol. In some embodiments, the one or more of a lithium salt is a lithium fluoride, a lithium chloride, a lithium bromide, a lithium iodide, a lithium acetate, a lithium tetraborate, a lithium carbonate, a lithium sulfate, a lithium nitrate, a lithium perchlorate, or a combination thereof. In some embodiments, the one or more of a lithium salt is a lithium chloride, a lithium bromide, a lithium iodide, or a combination thereof. In some embodiments, the one or more of a lithium salt is a lithium hydroxide. In some embodiments, the one or more of a lithium salt comprises a lithium chloride, a lithium bromide, or a lithium iodide, or a combination thereof. In some embodiments, the one or more of a lithium salt is a lithium chloride.

In some embodiments, the composition comprises about 0.1 M, about 0.5 M, about 1 M, about 1.5 M, about 2 M, about 2.5 M, about 3 M, about 3.5 M, about 4 M, about 4.5 M, about 5 M, about 5.5 M, or about 6 M lithium cation.

In some embodiments, the one or more of an alkylamine is a primary alkylamine, a secondary alkylamine, a tertiary alkylamine, or a combination thereof. In some embodiments, the one or more of an alkylamine is a tertiary alkylamine or a combination thereof. In some embodiments, the one or more of an alkylamine is trimethylamine, triethylamine, diisopropylethylamine, or a combination thereof. In some embodiments, the one or more of an alkylamine is triethylamine. In some embodiments, the composition comprises about 0.1% to about 20% w/w of the one or more of an alkylamine. In some embodiments, the composition comprises about 3% to about 15% w/w of the one or more of an alkylamine. In some embodiments, the composition comprises about 3% to about 10% w/w of the one or more of an alkylamine. In some embodiments, the composition comprises about 20 to about 32% w/w ammonia. In some embodiments, the composition is at least about 90% saturated with ammonia, or the composition is saturated with ammonia. In some embodiments, the composition comprises about 25 to about 32% w/w ammonia.

In some embodiments, the composition is selected from one of A-G:
A) an aqueous composition comprising about 0.1 M of a lithium salt, and about 20 to about 32% w/w ammonia;
B) an aqueous composition comprising about 1 M of a lithium salt, and about 20 to about 32% w/w ammonia;
C) an aqueous composition comprising about 2 M of a lithium salt, and about 20 to about 32% w/w ammonia;
D) an aqueous composition comprising about 3 M of a lithium salt, and about 20 to about 32% w/w ammonia;
E) an aqueous composition comprising about 4 M of a lithium salt, and about 20 to about 32% w/w ammonia;
F) an aqueous composition comprising about 5 M of a lithium salt, and about 20 to about 32% w/w ammonia; or
G) an aqueous composition comprising about 6 M of a lithium salt, and about 20 to about 32% w/w ammonia.

In some embodiments, either the lithium salt is lithium chloride, the ammonia is present at about 28 to about 32% w/w, or the lithium salt is lithium chloride and the ammonia is present at about 28 to about 32% w/w, optionally wherein the composition further comprises about 3% w/w diethylamine, about 10% w/w trimethylamine, about 10% w/w triethylamine, or about 40% w/w methylamine.

In some embodiments, either the lithium salt is lithium bromide, the ammonia is present at about 28 to about 32% w/w, or the lithium salt is lithium bromide and the ammonia is present at about 28 to about 32% w/w, optionally wherein the composition further comprises about 3% w/w diethylamine, about 10% w/w trimethylamine, about 10% w/w triethylamine, or about 40% w/w methylamine.

In some embodiments, either the lithium salt is lithium iodide, the ammonia is present at about 28 to about 32% w/w, or the lithium salt is lithium iodide and the ammonia is present at about 28 to about 32% w/w, optionally wherein the composition further comprises about 3% w/w diethylamine, about 10% w/w trimethylamine, about 10% w/w triethylamine, or about 40% w/w methylamine.

In some embodiments, either the lithium salt is lithium hydroxide, the ammonia is present at about 28 to about 32% w/w, or the lithium salt is lithium hydroxide and the ammonia is present at about 28 to about 32% w/w, optionally wherein the composition further comprises about 3% w/w diethylamine, about 10% w/w trimethylamine, about 10% w/w triethylamine, or about 40% w/w methylamine.

In some embodiments, provided herein is a method comprising contacting a solid support bound molecule with a lithium composition described herein.

In some embodiments, provided herein are methods of cleaving a molecule bound to a solid support (e.g., prepared by solid phase synthesis), comprising contacting the molecule bound to the solid support with a lithium composition described herein. In some embodiments, the molecule (e.g., oligonucleotide, peptide, or carbohydrate, etc.) bound to the solid support is covalently bound to the solid support, and on cleavage from the solid support the molecule includes an alcohol moiety at the point of cleavage. In some embodiments, the cleavage occurs over a time period that includes up to about 14 hours, or more, (e.g., up to about 1, 2, 4, 6, 8, 10, 12, 14, 16, or more hours) in a sealed container, e.g., a capped container, at a temperature of about 20-60° C., e.g., about 35, 40, 45, 50, or 55° C., in the presence of the lithum cation containing solution (e.g., containing LiCl, LiBr, LiI, or LiOH) and about 15-32% w/w ammonia, which solution optionally includes about diethylamine (e.g., about 3% w/w), triethylamine (e.g., about 10% w/w), methylamine (e.g., about 40% w/w), or a combination thereof. In some embodiments, the molecule is covalently linked to the solid support by a linker that includes a structure

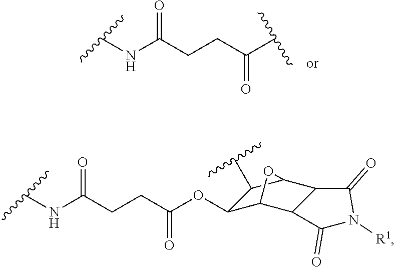

wherein R¹ is C₁₋₄ alkyl or C₆₋₁₀ aryl (e.g., methyl or phenyl). In some embodiments, the solution comprises: 1) an aqueous solution comprising lithium salt (e.g., about 2 or 4 M lithium salt, e.g., LiCl, LiBr, LiI, or LiOH) and about 24-35% by mass ammonia; or 2) an aqueous solution comprising lithium salt (e.g., about 2 or 4 M lithium salt, e.g., LiCl, LiBr, LiI, or LiOH), about 12-17% by mass (e.g., about 7-8 M) ammonia, and about 20% by mass methylamine; or 3) an aqueous solution comprising lithium salt (e.g., about 2 or 4 M lithium salt, e.g., LiCl, LiBr, LiI, or LiOH), about 22-32% by mass (e.g., about 13 M) ammonia, and about 10% by volume or mass triethylamine or diisopropylamine.

Provided herein is a molecule, prepared by a method described herein. In some embodiments the molecule comprises an oligonucleotide. In some embodiments, the molecule comprises a peptide. In some embodiments, the molecule comprises a carbohydrate.

In some embodiments, the molecule comprises one or more nucleoside or nucleotide residues, one or more amino acid residues, one or more saccharide residues, one or more polymers, or a combination thereof, and is prepared by the methods described herein.

Thus, the present disclosure relates to the efficient cleavage and deprotection method for 5′-bioconjugated oligonucleotides containing the POM protected (E)-vinylphosphonate or phosphate (originally, from Solid CPR II, as DMT-On) from solid support. Herein the cleavage and deprotection method may contain the lithium cation provided from various lithium sources such as lithium fluoride, lithium chloride, lithium bromide, and lithium iodide, lithium acetate, lithium tetraborate, lithium hydroxide, lithium carbonate, lithium sulfate, lithium nitrate, and/or lithium perchlorate, or their hydrate form. In some embodiments, the lithium source includes a lithium chloride. Concentration of lithium cation can be selected from about 0.1 M to about 6.0 M, which are corresponding to 0.1% to 40% depending on the molecular weight of lithium sources. In some embodiments, the lithium concentration is 1.0 M to 4.0 M. In some embodiments, the lithium concentration is 1.5 M to 2.5 M. In some embodiments, the lithium concentration is 2.0 M. Temperature can be selected from about RT to about 80° C. In some embodiments, the temperature is 30° C. to 70° C. In some embodiments, the temperature is 40° C. to 55° C. Reaction time can be selected from about 4 hours to about 48 hours. In some embodiments, the reaction time is 6 hours to 24 hours. In some embodiments, the reaction time is 8 hours to 14 hours. Solution quantity can be selected from about 100 μL/μmol to about 1.0 mL/μmol. In some embodiments, solution quantity is 100 μL/μmol to 500 μL/μmol. In some embodiments, solution quantity is 100 μL/μmol to 250 μL/μmol. Alkylamine additive can be included in the lithium reagent solution. In some embodiments, the alkylamine includes one or more of a tertiary alkylamine. In some embodiments, the alkylamine is triethylamine or diisopropylethylamine or a combination thereof. In some embodiments, the alkylamine quantity can be selected from about 0.1% to about 20% w/w. In some embodiments, the alkylamine quantity is 3% to 15% w/w. In some embodiments, the alkylamine quantity is 3% to 10% w/w. During the cleavage and deprotection, the container can be located in heat block without shaking or rocking or can be shaken or rocked at heating incubator with the given temperature (usually, shaking or rocking may accelerate the cleavage and deprotection, resulting in less impurities).

In some embodiments, provided herein are methods for the lithium assisted cleavage and deprotection of bioconjugated oligonucleotide from a solid support, said method comprising the step of treating the bioconjugated oligonucleotide bound to the solid support with the lithium cation and/or tertiary alkylamine in concentrated or diluted aqueous ammonia solution at ambient temperature for a period until there is no more found of the bioconjugated oligonucleotide bound to solid support tether.

### GalNAc Conjugation Linker

Since the discovery of GalNAc conjugates that bind to the asialoglycoprotein receptor (ASGPR), the targeted delivery of oligonucleotides to the liver hepatocytes has become a breakthrough approach in the field of oligonucleotide therapeutics. Introduction of GalNAc conjugates enables those oligonucleotides approved from regulatory authorities: Givlaari® (givosiran), Oxlumo® (Lumasiran), and Leqvio® (inclisiran). There has been known for several approaches to adopt the GalNAc conjugates. Alnylam pharmaceuticals developed the well-known tri-antennary GalNAc conjugation linkers. Arrowhead pharmaceuticals also developed their own multivalent GalNAc conjugation linkers using peptidyl backbone structures. Dicerna pharmaceuticals introduced the GalNAc conjugation linkers by incorporating GalNAc sugars attached to the extended region of oligonucleotides tetraloop (namely, GalXC compound). OliX Pharmaceuticals is known to utilize the stability enhanced peptidyl backbone of tri-GalNAc conjugation.

There have been several GalNAc conjugation linkers on market in the form of solid supports or phosphoramidites.

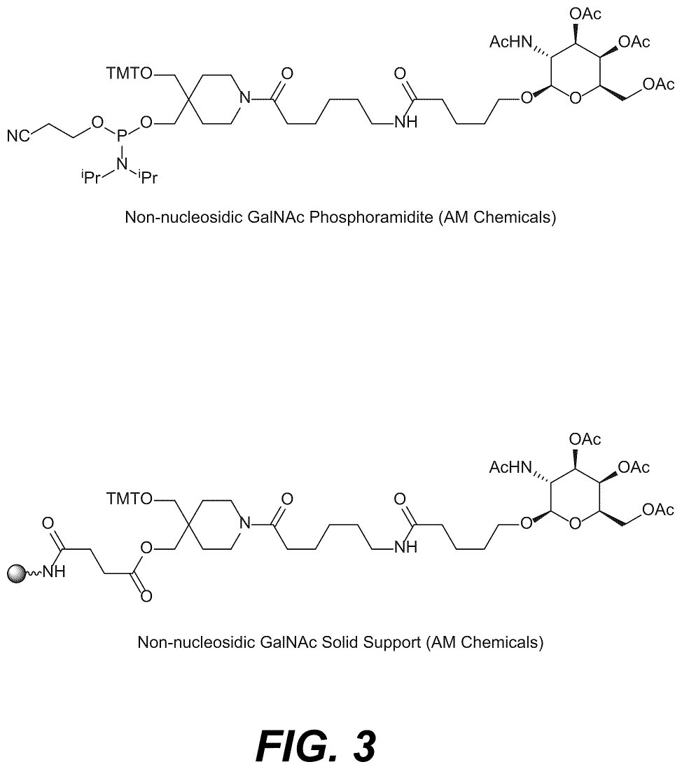

AM Chemicals developed the unique GalNAc conjugation linker composed of a piperidine ring to which a 1,3-diol structure is attached: non-nucleosidic GalNAc phosphoramidite (Product No. 51210) along with non-nucleosidic GalNAc Solid Support (Product No. 612101-612106) (U.S. Pat. No. 10,781,175 B2). For this line of products, a trimethoxytrityl group has been chosen instead of the more typical 4,4′-dimethoxytrityl (DMT) protecting group. The use of the more labile TMT groups ensures a rate of release equivalent to the regular DMT release. AM Chemicals has shown that these products are fully compatible with regular oligonucleotide synthesis. Furthermore, AM Chemicals demonstrated that non-nucleosidic GalNAc phosphoramidite can be used to prepare oligonucleotides with three consecutive GalNAc addition at either of 3′- and 5′-terminus. See FIG. 3 and FIG. 4.

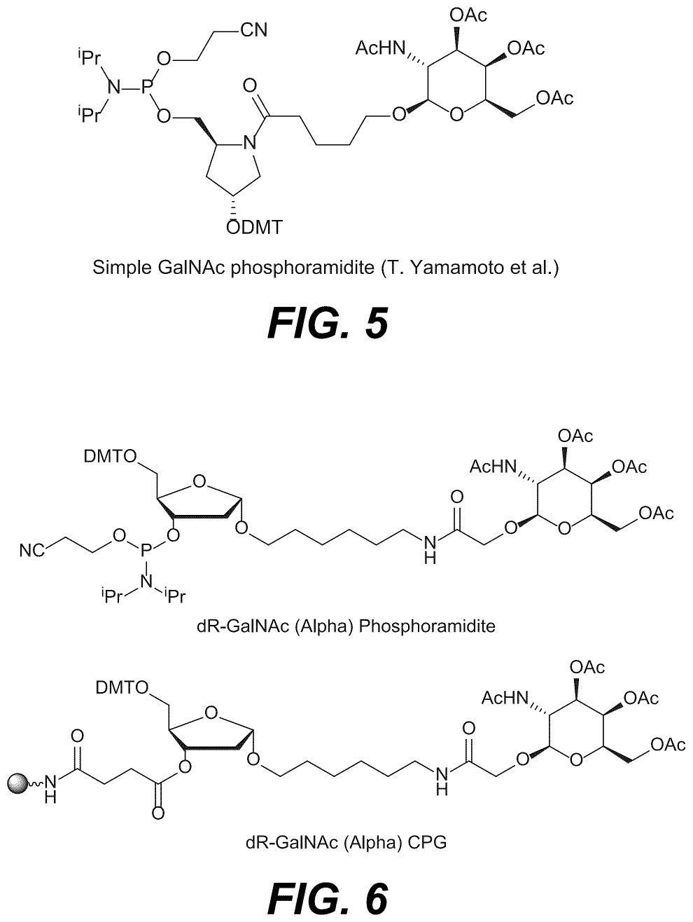

T. Yamamoto et al. (Bioorganic & Medicinal Chemistry, 2016, 24, 26-32) reported the effectiveness of modification at the 5′-terminus using monovalent GalNAc ligand on antisense oligonucleotides, where the synethetic method of GalNAc phosphoramidite was reported by K. G. Rajeev et al. (ChemBioChem, 2015, 16, 903). Up to five GalNAc monomer were added in a serial manner and it was shown that activity of the antisense oligonucleotide improved as the number of GalNAc increased. See FIG. 5.

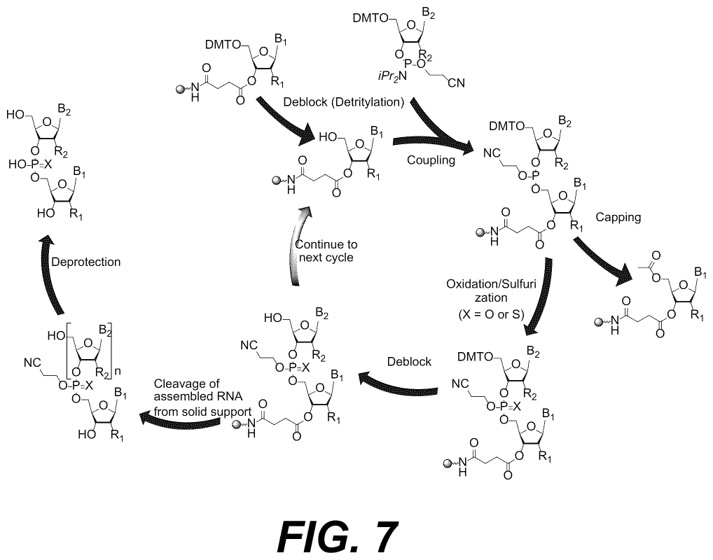

Synbase™-based GalNAc phosphoramidite is on market by Biosearch Technologies: GalNAc phosphoramidite (LK2568) and two equivalent GalNAc solid supports, with a choice between two different pore sizes (LK2440 and LK2489). This GalNAc monomer is advertised as used to build up flexible GalNAc clusters, varying in the number of GalNAc monomers and in their position within the oligonucleotides. This line of products have a deoxy ribose (dR) moiety which goes directly into the natural sugar phosphate backbone with little disruption of the duplex, meaning there is minimal effect on the Tm. Interestingly, modification on the dR backbone shows nuclease resistance with the alpha-monomer, but not with the beta-monomer (Y. C. Lee et al., 2008, Chapter 72. Interactions of oligosaccharides and glycopeptides with heptatic carbohydrate receptors. In carbohydrates in Chemistry and Biology: A Comprehensive Handbook. B. Ernst et al. eds. Wiley, pp 549-561). See FIG. 6 and FIG. 7.

### Solid Support

Oligonucleotide synthesis proceeds well on solid support such as controlled pore glass (CPG) or microporous polystyrene (MPPS) resin, which is covalently attached to the reactive amino groups in aminopropyl CPG, LCAA CPG, or aminomethyl MPPS.

Universal support where a non-nucleosidic linker is attached to the solid support is one of convenient and widely used materials for the oligonucleotide synthesis. A phosphoramidite respective to the 3′-terminal nucleoside residue is coupled to the universal solid support in the first synthetic cycle of oligonucleotide chain assembly using the standard oligonucleoside coupling protocol. After completion of oligonucleotide synthesis, the release of oligonucleotide occurs by the hydrolytic cleavage of a P—O bond that binds the 3′-hydroxyl group of nucleosides to the universal linker. In general, gaseous ammonia, methanolic ammonia, aqueous ammonium hydroxide, aqueous methylamine, or their mixture are well-known suitable reagents for the cleavage reaction. Special solid supports are also used for the attachment of desired functionality at the 3′-terminus of synthetic oligonucleotides.

Universal support I, first introduced in 1997 by Glen Research, popularly known as the McLean support, allows every oligonucleotide synthesis to take place on a single support without the need for a support for every desired 3′-nucleoside. The support found favor in high-throughput oligonucleotide synthesis platforms and became the universal support of choice for deprotecting using gaseous methylamine. However, since the phosphate elimination (dephosphorylation) reaction to yield a free 3′-hydroxyl group requires quite aggressive conditions, as outlined in Table 1B, this product has been somewhat limited in its applications. One problem was the fact that the product could not be used to prepare oligonucleotides with some of the most popular nucleoside analogues at the 3′-terminus since many of these are not compatible with the required aggressive dephosphorylation conditions because dephosphorylation is the slowest step (type 1: cleavage followed by deprotection and dephosphorylation).

Universal support III is unique among universal supports in that the dephosphorylation reaction occurs first and leads to cleavage from the solid support (type 2: dephosphorylation followed by cleavage and deprotection). This reaction occurs with mild anhydrous methanolic ammonia, or even with the UltraMild deprotection solution of anhydrous potassium carbonate in methanol. Oligonucleotide deprotection can then be achieved using any suitable procedure-AMA, ammonium hydroxide, and all other common methods. However, one issue with Universal linker III is that it is not readily compatible with gas phase cleavage and deprotection using anhydrous methylamine gas, and this strategy is used in many high-throughput situations. Without being bound by theory, it appears that the elimination of cyanoethyl protecting groups is favored in anhydrous methylamine gas. Once the cyanoethyl protecting group is eliminated to give the phosphodiester group, the amide-assisted dephosphorylation reaction to give the desired 3′-OH slowly stops. The yield of isolated oligonucleotide therefore suffers.

Isis Pharmaceuticals and Glen Research prepared the conformationally preorganized universal linker to accelerate the dephosphorylation reaction. By introducing a rigid bicyclic molecule on the support (namely, anchimeric assistance), the molecule's conformation would facilitate the formation of the cyclic phosphate transition state, thereby stimulating the rate of dephosphorylation. At present, Kinovate Life Sciences commercialized the UnySupport loaded on NittoPhase solid support and advertised it as outstanding yields and purity from lab to commercial scale at a highly competitive cost. UnySupport use may provide (1) elimination of the need for multiple succinates, (2) improved oligonucleotide quality, (3) consistency in batch-to-batch quality, (4) reduced cost through streamline of inventory to one solid support, (5) greater simplicity of inventory management and QC, and (6) greatly reducing regulatory requirements (no moiety from UnySupport integrated into API after cleavage).

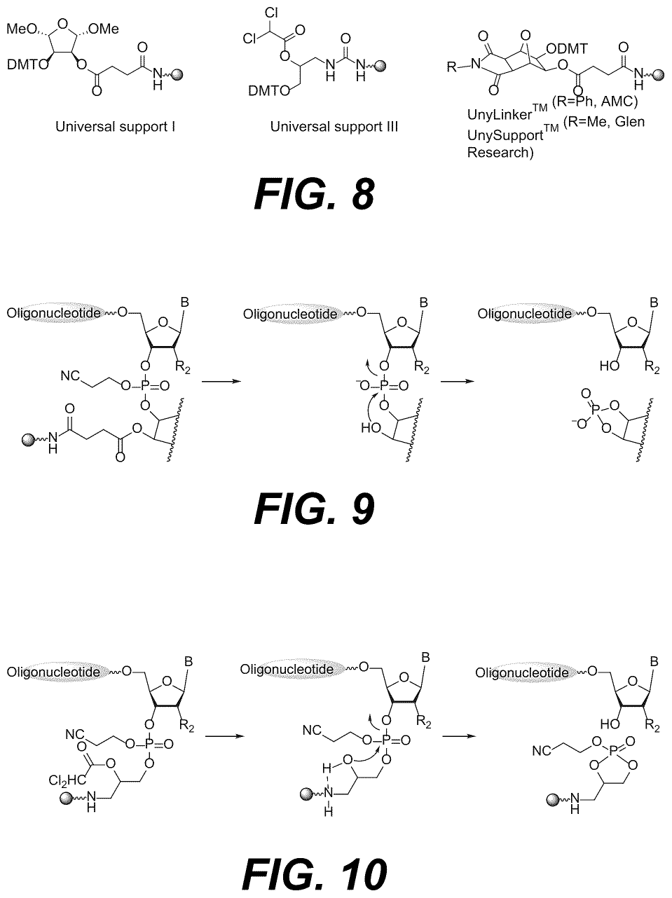

Other solid supports are described in S. Scott et al., 1994, A universal support for oligonucleotide synthesis, in Innovations and Perspective in Solid Phase Synthesis, 3^{rd} International Symposium, R. Epton, Eds, Mayflower Worldwide, pp 115-124; A. Azhayev et al., GEN, 2005, 25; A. P. Guzaev et al., J. Am. Chem. Soc., 2003, 125, 2380; R. K. Kumar et al., Tetrahedron, 2006, 62, 4528; U.S. Pat. No. 7,202,264 (Isis Pharmaceuticals). See FIG. 8.

FIG. 9 and FIG. 10 show deprotection and cleavage conditions during oligonucleotide synthesis. Table 1B shows elimination conditions for universal supports.

TABLE 1B
Elimination conditions for Universal supports

| Support | Reagent | Conditions |
|---|---|---|
| Universal | Ammonium hydroxide | 80° C., 8 h min. |
| support | Ammonium hydroxide/40% | 55° C., 17 h/80° C., 3 h min. |
| | methylamine (AMA) | |
| | 40% Methylamine | 55° C., 8 h |
| | 0.4M NaOH in | 80° C., 0.5 h/55° C., 3 h/RT, |
| | methanol/water (4:1) | 17 h |
| UnySupport | Ammonium hydroxide | 80° C., 2 h/55° C., 8 h |
| | Ammonium hydroxide/40% | 80° C., 0.5 h/65° C., |
| | methylamine (AMA) | 1 h/55° C., 2 h |
| | Methylamine gas | 65° C., 30 min at 30 psi |

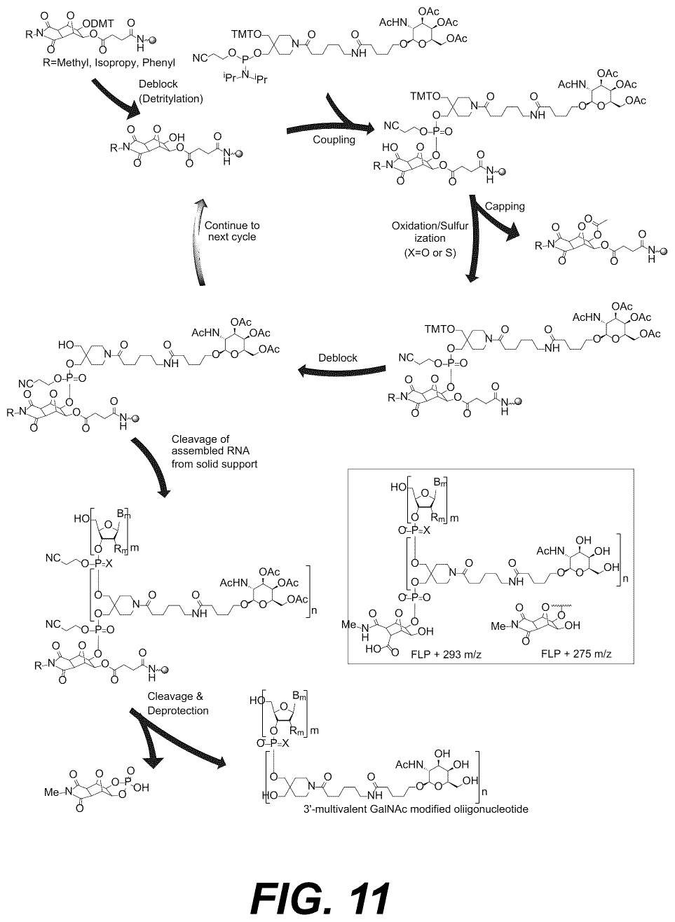

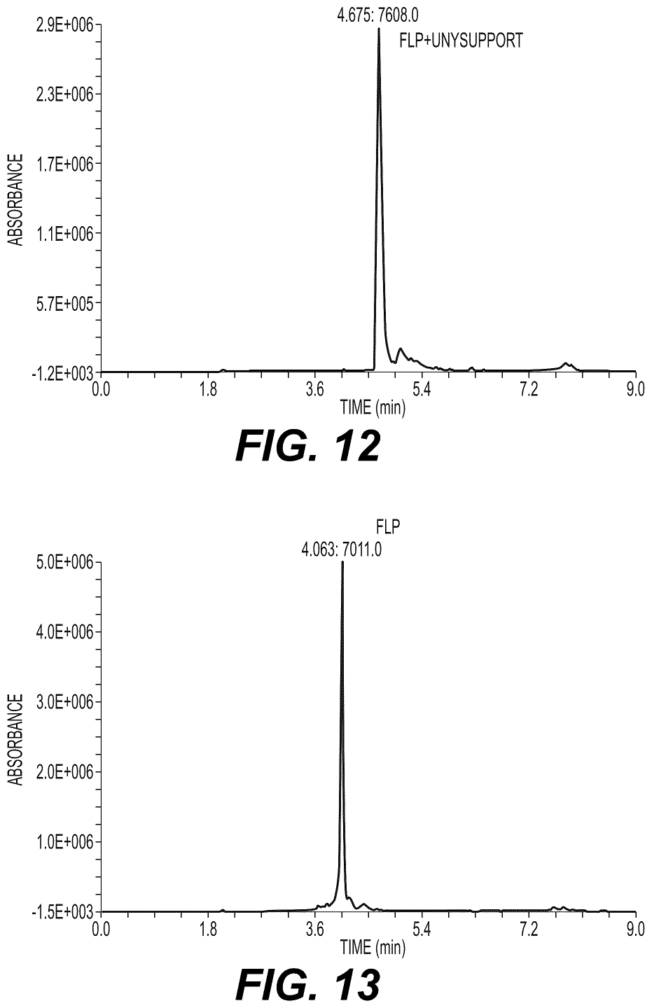

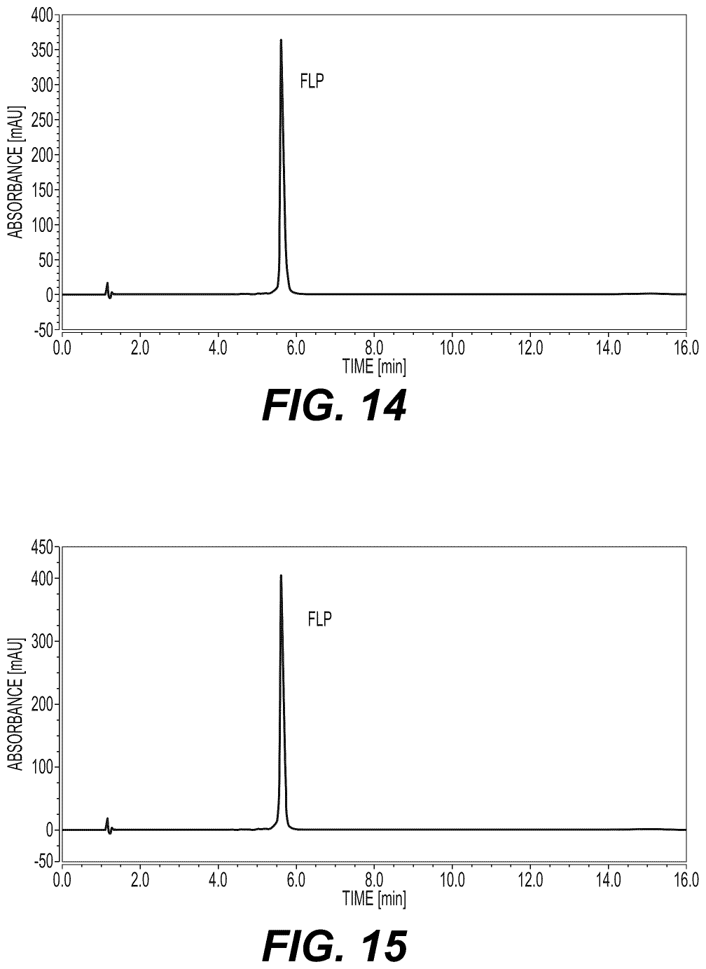

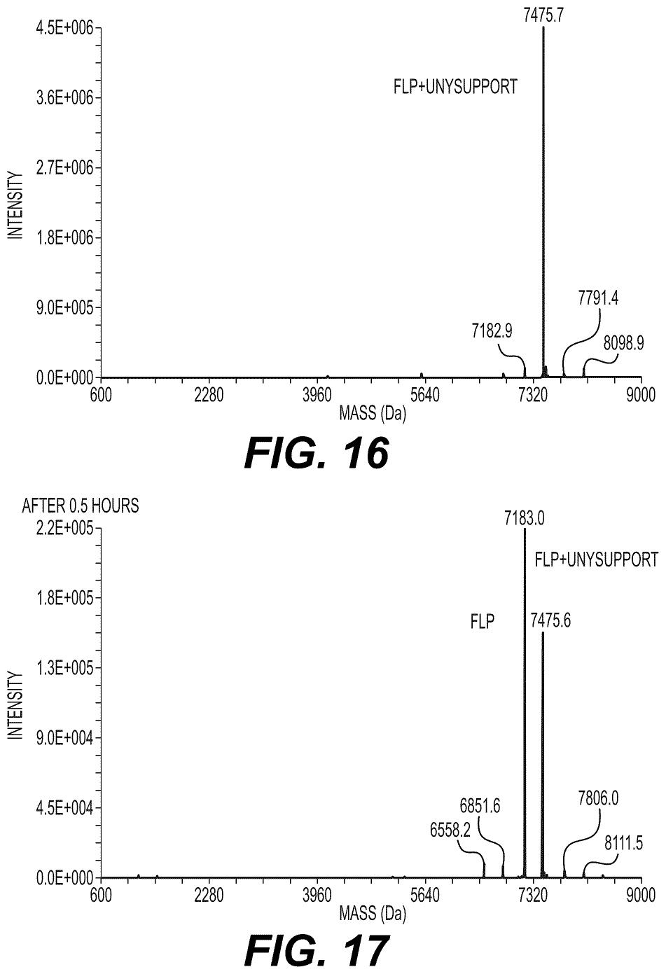

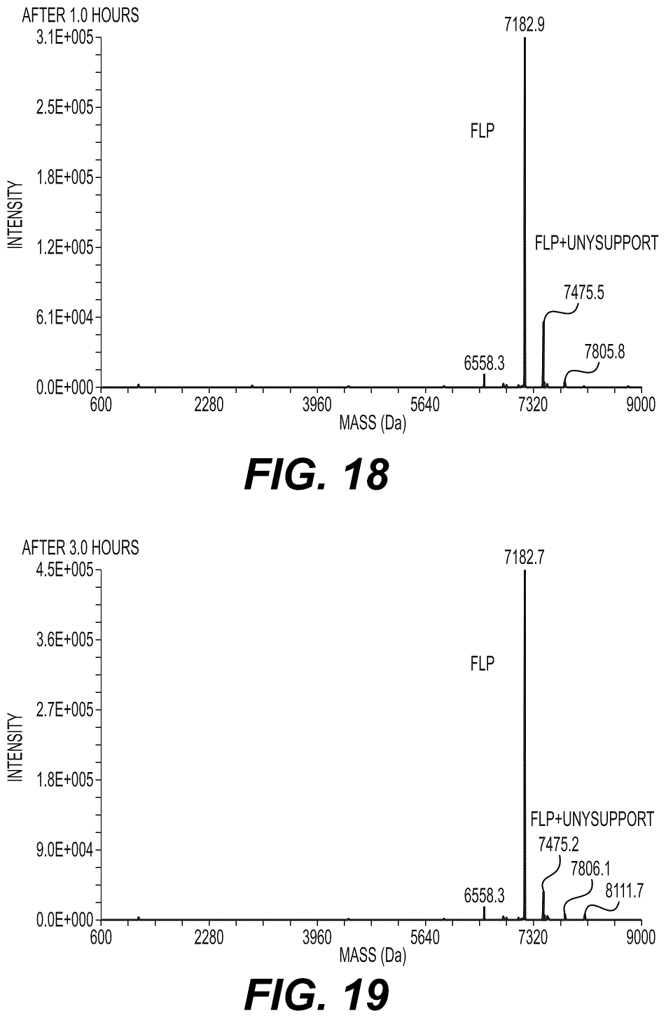

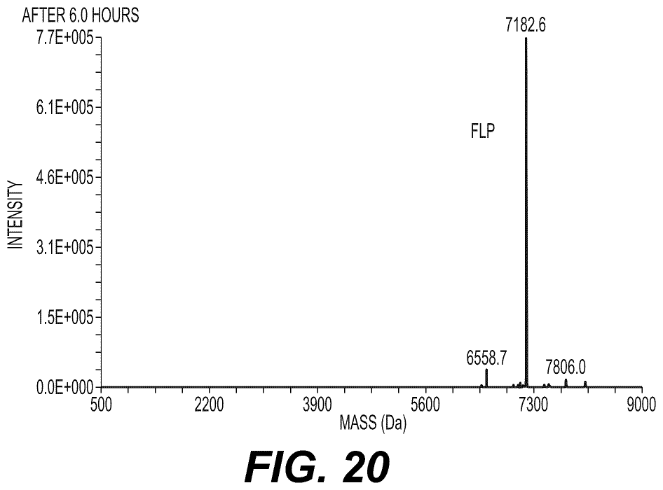

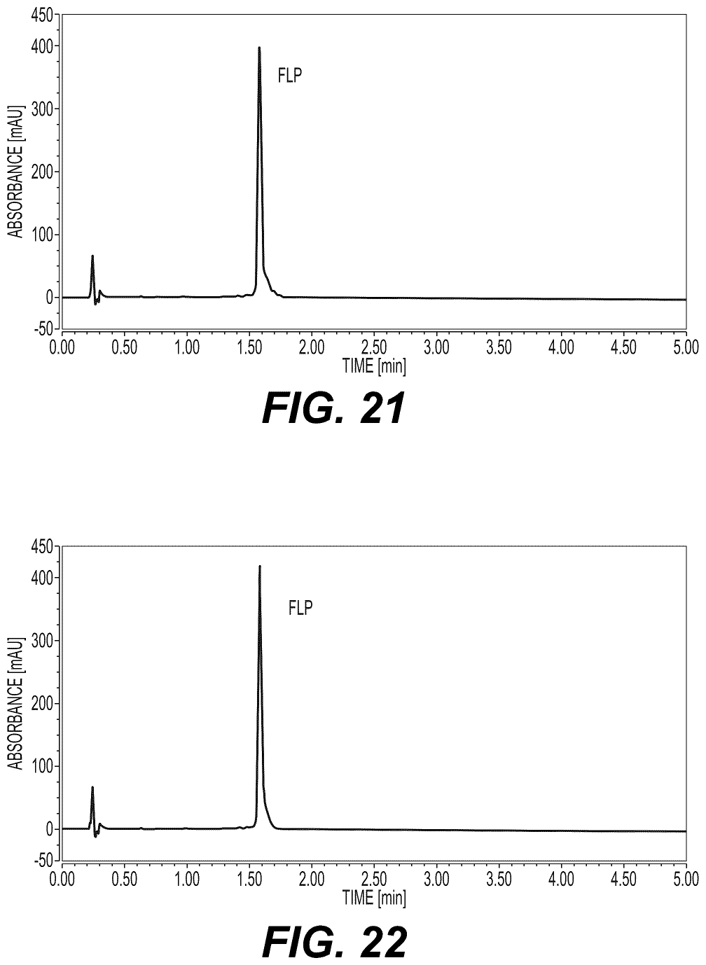

There are several issues in the process of oligonucleotide synthesis using non-nucleosidic GalNAc phosphoramidite (AM Chemicals) on UnySupport. Oligonucleotide is synthesized according to the oligonucleotide synthesis protocol, which is established for general nucleosidic phosphoramidites and non-nucleosidic phosphoramidites (e.g., CholTEG phosphoramidite). After cleavage and deprotection with AMA at 65° C. for 10 minutes or at RT for 24 hours, it is found that overall synthetic yield was unusually much lower, and oligonucleotide still contains UnySupport adduct via phosphate linkage. See FIG. 11.

Thus, also provided herein are methods for synthesis of oligonucleotides containing 3′-multivalent GalNAc conjugation linker comprising a modified synthetic protocol, an improved cleavage and deprotection method, and an improved purification method.

In some embodiments, provided herein is a method of oligonucleotide synthesis on UnySupport, comprising:
Deblocking by 10% dichloroacetic acid (DCA) in toluene;
Coupling with the first GalNAc amidite by 4,5-dicyanoimidazole (DCI) and with following (e.g., subsequent) nucleoside (e.g., modified) amidites by 5-ethylthio-1H-tetrazole (ETT);
Oxidation by (1S)-(+)-(10-camphorsulfonyl)-oxaziridine (CSO) for the first GalNAc coupling and by Iodine/Pyridine/THF for the following (e.g., subsequent) couplings;
Capping by a mixture of Cap Mix A and Cap Mix B (e.g., commercially available from Glen Research); and
Combination of synthetic process for 1^{st} coupling cycle and 2^{nd}/following coupling cycle.

In some embodiments, the method further comprises one or more of:
Basic treatment of the oligonucleotide using AMA at ambient temperature; or
Acidic treatment of the oligonucleotide using TFA at ambient temperature. In some embodiments, the oligonucleotide is purified by one or more of:
Oligonucleotide Purification Cartridge (OPC), Reverse Phase High Pressure Liquid Chromatography (RP-HPLC), or Anion Exchange HPLC (AEX-HPLC).

In some embodiments, the oligonucleotide is a 3′-tri-GalNAc conjugated oligonucleotide.

### Modified Synthesis of Oligonucleotide Containing 3′-Multivalent-GalNAc Conjugation Linker(s).

It is one object of this disclosure to provide the improved synthetic method of oligonucleotide containing 3′-multivalent-GalNAc conjugation linker(s). Coupling of the first monomer onto the UnySupport has extra detritylation using 10% DCA in toluene and double capping.

It is one object of this disclosure to provide the improved synthetic method of oligonucleotide containing 3′-multivalent-GalNAc conjugation linker(s). Incorporation of first GalNAc conjugation linker on UnySupport is achieved using non-nucleosidic GalNAc phosphoramidite from AM Chemicals by following reactions: deblock with 10% DCA in toluene, coupling with GalNAc phosphoramidite and DCI in Acetonitrile, oxidation with CSO ((1S)-(+)-(10-camphorsulfonyl)-oxaziridine), and double capping with a mixture of CapA (NMI (N-methylimidazole) in acetonitrile) and CapB (a mixture of acetic anhydride and 2,6-lutidine in acetonitrile).

It is one object of this disclosure to provide the improved synthetic method of oligonucleotide containing 3′-multivalent-GalNAc conjugation linker(s). Incorporation of second GalNAc conjugation linker or following (modified) nucleoside is achieved by following reactions: deblock with 3% DCA in toluene, coupling with GalNAc phosphoramidite or (modified) nucleoside phosphoramidite and ETT in Acetonitrile, oxidation with iodine in pyridine and water or DDTT in pyridine and Acetonitrile, and capping with a mixture of CapA (NMI in Acetonitrile) and CapB (a mixture of Acetic anhydride and 2,6-lutidine in Acetonitrile). No capping is performed for the final cycle.

It is one object of this disclosure to provide the improved synthetic method of oligonucleotide containing 3′-multivalent-GalNAc conjugation linker(s). The final detritylation is performed by using 3% DCA in toluene or skipped, depending on the selection of purification method

It is one object of this disclosure to provide the improved cleavage and deprotection method of oligonucleotide containing 3′-multivalent-GalNAc conjugation linker(s). After synthesis of oligonucleotide containing 3′-multivalent-GalNAc conjugation linker(s), solid support is dried by argon flow to the dryness. All protecting groups are removed by basic treatment using AMA solution. Solid support is filtered and washed with water. Collected filtrate is concentrated and treated by acid hydrolysis using aqueous trifluoroacetic acid solution. Solution is neutralized with triethylamine or TEAA buffer solution.

It is one object of this disclosure to provide the improved purification method of oligonucleotide containing 3′-multivalent-GalNAc conjugation linker(s). Oligonucleotide containing 3′-multivalent-GalNAc conjugation linker(s) is purified by anion exchange (AEX) or reverse phase (RP) column chromatography and desalted by dialysis-filtration. Collected oligonucleotide is concentrated in the final container.

The present disclosure relates to the improved synthetic method of oligonucleotide containing 3′-multivalent-GalNAc conjugation linker(s). Herein the solid support is selected from UnyLinker™ (R=Phenyl or Isopropyl, AM Chemicals) and UnySupport™ (R=Methyl, Glen Research or ChemGenes), where the backbone structure is composed of 5,6-dihydroxy-2-alkyl (R=methyl, isopropyl, phenyl) hexahydro-1H-4,7-epoxyisoindole-1,3(2H)-dione. One of 5,6-dihydroxyl group is protected with trityl group such as DMT (dimethoxytrityl) and the other is connected to the solid support through succinate linker. Additionally, UnyLinker or UnySupport can include the variances of R group by other alkyl groups such as isopropyl and aryl groups such as toluenyl or benzyl.

The present disclosure relates to the improved synthetic method of oligonucleotide containing 3′-multivalent-GalNAc conjugation linker(s). Herein the GalNAc amidite is non-nucleosidic GalNAc phosphoramidite (AM Chemicals), where the backbone structure is composed of 5-acetamido-2-(acetoxymethyl)-6-((5-((6-(4,4-bis (hydroxymethyl) piperidin-1-yl)-6-oxohexyl) amino)-5-oxopentyl) oxy) tetrahydro-2H-pyran-3,4-diyl diacetate. One primary hydroxyl group at piperidine is protected with trityl group such as TMT (trimethoxytrityl) and the other primary hydroxyl group at piperidine is phosphitylated with 2-cyanoethoxy diisopropylamino phosphaneyl group for the further coupling reaction.

The present disclosure relates to the improved synthetic method of oligonucleotide containing 3′-multivalent-GalNAc conjugation linker(s) linker. Herein the oligonucleotide is selected from moieties including, and not limited to, RNAs containing any of internucleotide linkage modifications, base modifications, sugar modifications, and non-radioactive labels, nucleic acid cross-linking. Also, the oligonucleotide is selected from RNAs containing any combination of those modifications.

The present disclosure relates to the improved synthetic method of oligonucleotide containing 3′-multivalent-GalNAc conjugation linker(s). Herein the oligonucleotide is selected from the oligonucleotide composed of 7 to 30 nucleotides.

The present disclosure relates to the improved synthetic method of oligonucleotide containing 3′-multivalent-GalNAc conjugation linker(s). Herein the 3′-multivalent-GalNAc conjugation linker(s) is selected from mono-GalNAc, di-GalNAc, tri-GalNAc, tetra-GalNAc, penta-GalNAc, and hexa-GalNAc in the sequential chain via phosphate or phosphorothioate internucleotide linkage.

The present disclosure relates to the improved synthetic method of oligonucleotide containing 3′-multivalent-GalNAc conjugation linker(s). Herein the improved synthetic method is oligonucleotide synthetic cycle composed of 4 chemical reactions: (1) deblock (detritylation), (2) coupling of amidite, (3) oxidation/sulfurization, and (4) capping. Details is as below:
(1) Deblock: the reaction to remove the protecting group on secondary alcohol (i.e., DMT) of UnySupport to provide the 5′-terminal hydroxyl group.
(2) Coupling of amidite: the reaction to couple the GalNAc amidite or nucleosidic (modified) amidite on the 5′-terminal hydroxyl group of UnySupport in the presence of activator.
(3) Oxidation/sulfurization: the reaction to oxidize or sulfurize the internucleotide linkage.
(4) Capping: the reaction to protect the unreacted 5′-terminal hydroxyl group during the coupling of amidite.

The present disclosure relates to the improved synthetic method of oligonucleotide containing 3′-multivalent-GalNAc conjugation linker(s). Deblock is performed by acidic solution selected from dichloroacetic acid, trichloroacetic acid, and trifluoroacetic acid in aprotic organic solvent such as, and not limited to, acetonitrile, N,N-dimethylformamide, toluene, tetrahydrofuran, dichloromethane, 1,2-dichloroethane. Deblock is performed by dichloroacetic acid in aprotic solvent such as and not limited to acetonitrile, N,N-dimethylformamide, toluene, tetrahydrofuran, dichloromethane, 1,2-dichloroethane. Deblock is performed by dichloroacetic acid in toluene. In some embodiments, deblock is performed by 10% dichloroacetic acid in toluene for the first 3′-GalNAc coupling reaction cycle (Removal of DMT on UnyLinker or UnySupport) and 3% dichloroacetic acid in toluene for the following coupling reaction cycles. Deblock is performed by 10% dichloroacetic acid in toluene for the first 3′-GalNAc coupling reaction cycle in 8 column volume (CV) and 3% dichloroacetic acid in toluene for the following coupling reaction cycles in 8 column volume (CV). Incomplete deblock may occur when deblock is performed by lower concentration or lower amount of DCA in toluene to result in low yield of oligonucleotide synthesis.

The present disclosure relates to the improved synthetic method of oligonucleotide containing 3′-multivalent-GalNAc conjugation linker(s). Coupling of amidite is performed by GalNAc amidite or nucleoside amidite in the presence of activator. GalNAc amidite is selected from the product of AM Chemicals (Product No. 51210). Nucleoside amidite is selected from any of nucleoside amidites. Activator is selected from activators such as, and not limited to, tetrazole, 4,5-dicyanoimidazole (DCI), 5-(Ethylthio)-1H-tetrazole (ETT), and 5-(benzylthio)-1H-tetrazole (BTT). Aprotic solvent selected from solvents such as, and not limited to, acetonitrile, N,N-dimethylformamide, toluene, tetrahydrofuran, dichloromethane, 1,2-dichloroethane. In some embodiments, activator is 4,5-dicyanoimidazole (DCI) in acetonitrile for the first 3′-GalNAc coupling reaction cycle and 5-(Ethylthio)-1H-tetrazole in acetonitrile for the following coupling reaction cycles. In some embodiments, activator is 1 M DCI in acetonitrile for the first 3′-GalNAc coupling reaction cycle and 0.6 M ETT in acetonitrile for the following coupling reaction cycles. A % volume of activator is selected from 10% to 90% by volume. A % volume of activator, 1 M DCI in acetonitrile for the first 3′-GalNAc coupling reaction cycle is 70% and 0.6 M ETT in acetonitrile for the following coupling reaction cycles is 60%. All coupling reaction utilizes the 2-10 equivalences of amidite with a given % volume of activator for each coupling cycle. In some embodiments, 2 equivalences of amidite with a given % volume of activator is utilized for the coupling reaction. The first 3′-GalNAc coupling reaction time is elongated by 2.5 times, compared to the following GalNAc and nucleoside coupling reaction time.

Amidite coupling efficiency is mostly dependent on the combination of amidite and activator. Solubility and pKa of activator mostly contribute on the reactivity of GalNAc amidite and nucleoside amidite. Intermediate from the first 3′-GalNAc coupling reaction is relatively unstable under the strong acidic condition. When BTT or ETT was used for the coupling reaction, final yield of oligonucleotide synthesis was lower than when DCI was used. After oxidation, this intermediate became more stable to acidic activators. For the following coupling reaction using nucleoside amidite, ETT was efficiently working as activator because its acidity (pKa 4.3) is slightly higher than DCI (pKa 5.2). This is because the high acidity of the activator can help to protonate the nucleophilic oxygen on the incoming monomer, making it more reactive and increasing the likelihood of a successful coupling reaction.

The present disclosure relates to the improved synthetic method of oligonucleotide containing 3′-multivalent-GalNAc conjugation linker(s). Oxidation for phosphate is performed by oxidizing reagent such as, and not limited to, (1S)-(+)-(10-camphorsulfonyl) oxaziridine (CSO), iodine in pyridine and water solution, hydrogen peroxide, and di-tert-butoxy peroxide. Aprotic solvent is selected from solvents such as, and not limited to, acetonitrile, N,N-dimethylformamide, toluene, tetrahydrofuran, dichloromethane, 1,2-dichloroethane. In some embodiments, oxidizing reagent is CSO in acetonitrile for the first 3′-GalNAc coupling reaction cycle and iodine in pyridine and water (9:1 v/v) solution for the following coupling reaction cycles. More In some embodiments, oxidizing reagent is 0.1 M CSO in acetonitrile for the first 3′-GalNAc coupling reaction cycle and 50 mM iodine in pyridine and water (9:1 v/v) solution for the following coupling reaction cycles. Amount of oxidizing reagent is 1 equivalent to 10 equivalents for amidite. In some embodiments, amount of oxidizing reagent is 5 equivalents of 0.1 M CSO in acetonitrile for the first 3′-GalNAc coupling reaction cycle and 4 equivalents of 50 mM iodine in pyridine and water (9:1 v/v) solution for the following coupling reaction cycles. Oxidation for phosphorothioate is also performed by thiolation reagent such as, and not limited to, 3H-1,2-benzodithiol-3-one-1,1-dioxide, tetraethyhiuram disulfide, phenylacetyl disulfide, dibenzoyl tetrasulfide, bis-(O,O-diisopropoxyphosphinothioyl) disulfide, benzyltriethylammonium tetrathiomolybate, bis-(p-toluenesulfonyl) disulfide, 3-ethoxy-1,2,4-dithiazoline-5-one (EDITH), 1,2,4-dithiazolidine-3,5-dione, 3-amino-1,2,4-dithiazole-5-thione, 3-methyl-1,2,4-dithiazolin-5-one, 3-phenyl-1,2,4-dithiazoline-5-one, and 3-[(dimethylaminomethylene) amino]-3H-1,2,4-dithiazole-5-thione (DDTT), in aprotic solvent selected from solvents such as, and not limited to, acetonitrile, N,N-dimethylformamide, toluene, tetrahydrofuran, dichloromethane, 1,2-dichloroethane. In some embodiments, oxidizing reagent is DDTT in 1:1 (v/v) mixture of acetonitrile and pyridine. More In some embodiments, oxidizing reagent is 0.06 M DDTT in 1:1 (v/v) mixture of acetonitrile and pyridine. Amount of oxidizing reagent is 1-12 equivalents of DDTT in 1:1 (v/v) mixture of acetonitrile and pyridine for all coupling reaction cycles. Perferably, Amount of oxidizing reagent is 6 equivalents of DDTT in 1:1 (v/v) mixture of acetonitrile and pyridine for all coupling reaction cycles.

Oxidation is usually performed with anhydrous CSO in acetonitrile or hydrous iodine in a mixture of pyridine and water. The intermediate from the first 3′-GalNAc coupling reaction is relatively unstable under the strong hydrated acidic condition using iodine in a mixture of pyridine and water. Long incubation in iodine-based oxidizing reagent disclosed the decomposition of this 3′-mono-GalNAc adduct on UnySupport. So, intermediate for the first 3′-GalNAc coupling reaction is oxidized by CSO in acetonitrile to result in much less decomposition. After oxidation, this intermediate became more stable to iodine-based oxidizing reagent. For the following coupling reaction using nucleoside amidite, iodine in a mixture of pyridine and water was efficiently working as oxidizing reagent.

The present disclosure relates to the improved synthetic method of oligonucleotide containing 3′-multivalent-GalNAc conjugation linker(s). In order to avoid deletion of sequence, a few unreacted 5′-hydroxyl groups on the resin-bound nucleotides are needed to be capped. Capping is performed by a mixed solution of Capping reagents, CapA and CapB, where CapA is a solution of 20% N-methylimidazole in acetonitrile (v/v) and CapB is a solution of 20% acetic anhydride, 30% 2,6-lutidine in acetonitrile (v/v/v) for all coupling reaction cycles.

The present disclosure relates to the improved synthetic method for making oligonucleotide containing 3′-multivalent-GalNAc conjugation linker(s). Wash is performed by aprotic solvent such as, and not limited to, acetonitrile, N,N-dimethylformamide, toluene, tetrahydrofuran, dichloromethane, 1,2-dichloroethane. In some embodiments, wash is performed by acetonitrile. Amount of wash is 1-16 column volume (CV) of acetonitrile for all coupling reaction cycles. In some embodiments, amount of wash is 4-8 column volume of acetonitrile for all coupling reaction cycles.

The present disclosure relates to the improved synthetic method of oligonucleotide containing 3′-multivalent-GalNAc conjugation linker(s). Cleavage and deprotection is composed of basic reagent treatment and acidic reagent treatment. Basic reagent is selected from reagents such as, and not limited to, aqueous ammonium hydroxide, aqueous methylamine, ethanolic methylamine, diethylamine and potassium carbonate in methanol. Basic reagent is selected from any combination of basic reagents. In some embodiments, basic reagent is AMA (1:1 v/v mixture of aqueous ammonium hydroxide and aqueous methylamine). Acidic reagent is selected from reagents such as, and not limited to, dichloroacetic acid, trichloroacetic acid, and trifluoroacetic acid, in organic solvent or water. Acidic reagent is 1-10% trifluoroacetic acid in water. In some embodiments, acidic reagent is 2.5% trifluoroacetic acid in water.

In general, the first coupling reaction is usually performed with nucleoside amidite for the 3′-unmodified oligonucleotide synthesis to result in phosphate internucleotide linkage between UnySupport and 3′-hydroxyl group on nucleoside, where the 3′-hydroxyl group is secondary alcohol on sugar moiety of nucleoside. This phosphate linkage is readily cleaved by any basic reagents such as aqueous ammonium hydroxide, aqueous methylamine, and AMA (a 1:1 (v/v) mixture of aqueous ammonia and aqueous methylamine) to result in the formation of cyclophosphate with the aid of a conformationally preorganized UnySupport. Herein the secondary alcohol is not much considered very important so far. However, when the first coupling reaction is performed with GalNAc amidite from AM Chemicals for the 3′-GalNAc conjugated oligonucleotide synthesis, the resulting internucleotide phosphate linkage is formed between UnySupport and primary alcohol of GalNAc conjugation linker. After basic cleavage and deprotection using AMA, the aberrant mass of oligonucleotide was observed by increased mass of m/z +275 (N-methyl pyrrolidine dione ring-closed adduct) or +293 (N-methylamido carboxylate ring-open adduct). This proved that the oligonucleotide still contained the UnySupport by phosphate linkage. Interestingly, this phosphate linkage is not readily cleaved by any other basic reagent. Longer exposure under higher temperature didn't show any efficient method to cleave this phosphate linkage between UnySupport and GalNAc conjugation linker. Herein, this disclosure found that the acidic treatment resulted in the cleavage of UnySupport and phosphate linkage from oligonucleotide. 2.5% trifluoroacetic acid in water was found to be the most efficient to cleave the UnySupport adduct from oligonucleotide. Various concentration of dichloroacetic acid and trichloroacetic acid were tested to result in incomplete cleavage of UnySupport adduct. Lower concentration of trifluoroacetic acid in water took a longer time to the completion, and higher concentration of trifluoroacetic acid in water caused a slight decomposition of oligonucleotide. At present, the most preferred condition was 2.5% trifluoroacetic acid in water at RT for 2˜6 hour to result in completion of cleavage.

The present disclosure relates to the improved synthetic method of oligonucleotide containing 3′-multivalent-GalNAc conjugation linker(s). Cleavage and deprotection is performed at ambient temperature from about 10° C. to 80° C. Cleavage and deprotection can be performed at 10° C. Cleavage and deprotection can be performed at room temperature. Cleavage and deprotection can be performed at 40° C. Cleavage and deprotection can be performed at 65° C. Depending on the temperature of cleavage and deprotection, the duration can be adjusted to the completion. The temperature would be different for basic reagent treatment and acidic reagent treatment. In some embodiments, the basic reagent treatment prefers the higher temperature and acidic reagent treatment prefers the lower temperature to comply with the cleavage and deprotection profile regarding the impurities.

The present disclosure relates to the improved synthetic method of oligonucleotide containing 3′-multivalent-GalNAc conjugation linker(s). Purification of oligonucleotide is performed by oligonucleotide purification column (OPC), reverse phase column chromatography (RP-CC), anion exchange column chromatography (AEX-CC), followed by desalting process. To assess quality, crude and final products are analyzed using ion exchange chromatography and a high throughput characterization system with liquid chromatography-mass spectrometry (deconvolution of oligonucleotides).

The present disclosure relates to the improved synthetic method of oligonucleotide containing 3′-multivalent-GalNAc conjugation linker(s). Purification of oligonucleotide is performed by OPC. Oligonucleotide is loaded on OPC and washed by 1-15% acetonitrile in buffer solution to remove impurities. Then, oligonucleotide is eluted higher concentration of acetonitrile in buffer solution, followed by desalting to result in high purity of oligonucleotide.

The present disclosure relates to the improved synthetic method of oligonucleotide containing 3′-multivalent-GalNAc conjugation linker(s). Purification of oligonucleotide is performed by RP-CC, which follows the general oligonucleotide purification method.

The present disclosure relates to the improved synthetic method of oligonucleotide containing 3′-multivalent-GalNAc conjugation linker(s). Purification of oligonucleotide is performed by AEX-CC, which follows the general oligonucleotide purification method.

The present disclosure relates to the improved synthetic method of oligonucleotide containing 3′-multivalent-GalNAc conjugation linker(s). This method achieves overall synthetic yield of oligonucleotide containing 3′-multivalent-GalNAc conjugation linker(s) more than 50%. Overall synthetic yield of oligonucleotide is less than 50% if the general method is applied using 3% DCA in toluene for deblock, 0.6 M ETT as activator for coupling reaction, iodine in a mixture of pyridine and water for oxidation, and basic and acidic cleavage and deprotection. Moreover, the purity of oligonucleotide containing 3′-multivalent-GalNAc conjugation linker(s) yields greater than 85%, otherwise typically less than 70% after basic and acidic cleavage deprotection. This means there is not required of further purification using OPC, reverse phase column chromatography, or anion exchange column chromatography. After desalting, highly purified oligonucleotide containing 3′-multivalent-GalNAc conjugation linker(s) can be easily obtained.

The following examples further illustrate aspects of the present disclosure. However, they are in no way a limitation of the teaching or disclosure as described herein.

### EXAMPLES

The present methods are described more specifically, with the aid of various reaction scheme and examples. These specific schemes and examples should be constructed as illustrating the claimed methods, and not as limiting the same.

### General Procedures for the Forward Synthesis of Bioconjugated Oligonucleotides

Oligonucleotides were synthesized on Dr. Oligo 48, MerMade 12, or Oligo Pilot 100 synthesizer, depending on the scale. The solid support used was UnySupport 500 Å CPG (79 μmol/g) from ChemGenes. Non-nucleosidic phosphoramidites were purchased from AM Chemicals. Nucleosidic phosphoramidites were purchased from ChemGenes. Clustered tri-GalNAc conjugation was initially introduced on Amino C7 modifier CPG by OliX US. Saturated or unsaturated fatty acids were introduced by post-synthetic modification on Amino C6 modifier or initially introduced on Amino C7 modifier CPG by OliX US. Cyanine3 amidite was purchased from Lumiprobe. Chemical reagents and solvents were all commercially purchased and used as is.

During the oligonucleotide synthesis in general, deblock was a solution of 3% dichloroacetic acid in dichloromethane. Activation utilized a solution of 0.6 M 5-(S-ethylthio)-1H-tetrazole in acetonitrile. The phosphoramidite solutions were 0.075 to 0.15 M in anhydrous acetonitrile or 1:1 (v/v) of acetonitrile and N,N-dimethylformamide. The oxidizing reagent was 0.02 iodine in tetrahydrofuran (THF)/Pyridine/water (7:2:1 v/v/v). N,N-Dimethyl-N′-(3-thioxo-3H-1,2,4-dithiazole-5-yl) methanimidamide (DDTT) 0.06 M in 1:1 (v/v) mixture of pyridine and water was used as the sulfurizing reagent. Bioconjugated oligonucleotides were synthesized by modified synthesis cycles, based on those optimized with the instruments. Final detritylation was varied on their purification method or post-synthetic modification thereof. Post-synthetic modification was achieved on Amino C6 modifier after oligonucleotide reverse synthesis, utilizing the consequent amide coupling reaction with given substrates such as amino acids and various spacers (GABA, AEEA (or AEA), AEEP, TEG, HEG etc. where GABA is gamma amino butyric acid, AEAA is aminoethoxyacetic acid, AEEP is aminoethoxyethoxypropanoic acid, TEG is tetraethylene glycol, and HEG is hexaethylene glycol).

### General Cleavage and Deprotection Procedure for the Bioconjugated Oligonucleotide

After drying by argon flow, the 1 μmol of oligonucleotide on UnySupport CPG (15˜25 mg) was removed from the synthesis column and transferred into a 2 mL conical screw capped tube. 100 μL of fresh-made 2 M or 4 M lithium chloride in concentrated (28-30%) aqueous ammonia solution was added into the tube containing oligonucleotide on UnySupport CPG, where the CPG must be completely immersed in solution. Tube was vigorously vortexed more than 10 seconds to be completely mixed and wet with solution. Tube, capped, was placed in pre-heated shaker or heat block as per the designed temperature and reaction time. Cleavage and deprotection progress was monitored by high-throughput mass spectroscopy. After completion of the cleavage and deprotection, tube was cooled down to room temperature and placed in ice bath for 5 minutes. 200 μL of quenching reagent (1 M triethylammonium acetate (TEAA), pH 4.5) was added into a tube in ice bath. Tube was vigorously vortexed more than 10 seconds to be completely mixed. After centrifugation to spin down the CPG at 4000 RPM for 1 minute, supernatant was decanted into a new tube. After 3 times of repeated rinse with water, centrifugation and decantation, the resulting tube was stored at 4° C. for the further purification process with RP- or AEX-HPLC or desalting process with tangential flow filtration (TFF).

Preparation of certain examples of lithium solutions used herein includes the following: 1) desired molar amount of lithium salt is added to concentrated (about 24-35% by mass) aqueous ammonia solution and dissolved to provide an aqueous solution comprising lithium salt (e.g., about 2 or 4 M lithium salt, e.g., LiCl, LiBr, LiI, or LiOH) and about 24-35% by mass ammonia; or 2) desired molar amount of lithium salt is added to concentrated (about 24-35% by mass) aqueous ammonia solution and dissolved, which lithium salt solution is combined 1:1 by volume with 40% by mass aqueous methylamine to provide an aqueous solution comprising lithium salt (e.g., about 2 or 4 M lithium salt, e.g., LiCl, LiBr, LiI, or LiOH), about 12-17% by mass (e.g., about 7-8 M) ammonia, and about 20% by mass methylamine; or 3) desired molar amount of lithium salt is added to concentrated (about 24-35% by mass) aqueous ammonia solution and dissolved, which lithium salt solution is combined 9:1 by volume or mass with tertiary amine (e.g., triethylamine or diisopropylamine) to provide an aqueous solution comprising lithium salt (e.g., about 2 or 4 M lithium salt, e.g., LiCl, LiBr, LiI, or LiOH), about 22-32% by mass (e.g., about 13 M) ammonia, and about 10% by volume or mass triethylamine or diisopropylamine.

### Example 1A. Selection of Metal Cation Sources in Chloride Complex

1 μmol of tri-GalNAc bioconjugated oligonucleotide on UnySupport CPG (15˜25 mg) was dried under argon flow and treated with 1.0 mL of concentrated (28-30%) aqueous ammonia solution with 0.1 M, 1.0 M, 2.0 M and 4.0 M concentration of various metal chloride complex at 55° C. for 14 hours. Ratio of two molecular weight corresponding to the fully cleaved oligonucleotide and UnySupport tethered oligonucleotide was analyzed by HT/LCMS. As a result, the lithium chloride was selected for further experiments. See the Table 1A.

### Example 2A. Selection of Lithium Cation Sources from Various Salts

1 μmol of tri-GalNAc bioconjugated oligonucleotide on UnySupport CPG (15˜25 mg) was dried under argon flow and treated with 1.0 mL of concentrated (28-30%) aqueous ammonia solution with 2.0 M concentration of various lithium salts at 55° C. for 10-14 hours. Ratio of two molecular weight corresponding to the fully cleaved oligonucleotide and UnySupport tethered oligonucleotide was analyzed by High throughput liquid chromatography mass spectrometry (HT/LCMS). As a result, the lithium chloride was selected for further experiments. See the Table 2A.

### Example 3A. Selection of Optimal Cleavage and Deprotection Condition with Lithium Chloride

1 μmol of tri-GalNAc bioconjugated oligonucleotide on UnySupport CPG (15˜25 mg) was dried under argon flow and treated with 100-1000 μL of concentrated (28-30%) aqueous ammonia solution with 1.0 M to 6.0 M concentration of lithium chloride at 40° C. or 55° C. until the cleavage and deprotection was complete. Reaction progress and related impurities were analyzed by HT/LCMS. As a result, the optimal condition was selected as 4.0 M lithium chloride in concentrated (28-30%) aqueous ammonia solution at 40° C. for 24 hours or at 55° C. for 8-16 hours. The quantity might be selected from 100 to 500 μL/μmol. See the Table 3A.

### Example 4A. Scale-Up of Lithium Assisted Cleavage and Deprotection (320 μmol)

320 μmol of tri-GalNAc bioconjugated oligonucleotide on UnySupport CPG (about 6 g) was dried under argon flow and treated with 32, 64 mL, or 160 mL of 4.0 M lithium chloride in concentrated (28-30%) aqueous ammonia solution at 55° C. until the cleavage and deprotection was complete. Reaction progress and related impurities were analyzed by HT/LCMS. As a result, the cleavage and deprotection was complete within 9 to 14 hours according to the reaction volume. Similar results were obtained form 4 different sequences containing tri-GalNAc bioconjugation under the above-mentioned conditions. See the Table 4A.

### Example 5A. Application of Lithium Assisted Cleavage and Deprotection to Various Oligonucleotides Containing Non-Nucleosidic Modifier from AM Chemicals

10 μmol of mono-cholesterol conjugated oligonucleotide on UnySupport CPG was dried under argon flow and treated with 2 mL of 4.0 M lithium chloride in concentrated (28-30%) aqueous ammonia solution at 55° C. for 10 hours. The same condition was applied to other oligonucleotides containing mono-DL-tocopherol or mono-palmitic acid. 10 μmol of mono-linoleic acid conjugated oligonucleotide on UnySupport CPG was dried under argon flow and treated with 5 mL of 4.0 M lithium chloride in concentrated (28-30%) aqueous ammonia solution at 45° C. for 12 hours. The same condition was applied to other oligonucleotide containing mono-linolenic acid. Reaction progress and related impurities were analyzed by HT/LCMS. Lithium assisted cleavage and deprotection showed the completion of cleavage and deprotection. See Table 5A.

### Example 6A. Application of Lithium Assisted Cleavage and Deprotection to Various Reversely Synthesized Oligonucleotides Containing Post-Synthetically Modified Fatty Acids Conjugation

10 μmol of 3′-mono-palmitic acid conjugated oligonucleotide on UnySupport CPG at 5′ was dried under argon flow and treated with 5 mL of 4.0 M lithium chloride in concentrated (28-30%) aqueous ammonia solution at 55° C. for 8-10 hours. Reaction progress and related impurities were analyzed by HT/LCMS. Results showed the completion of cleavage and deprotection with lower impurity profile of PO impurity. See Table 6A.

### Example 7A. Application of Lithium Assisted Cleavage and Deprotection to 5′-POM Protected (E)-Vinylphosphonate Bioconjugated Oligonucleotides

1 μmol of 5′-POM protected (E)-vinylphosphonate bioconjugated oligonucleotides on UnySupport CPG was dried under argon flow and treated with 0 to 6.0 M lithium chloride in concentrated (28-30%) aqueous ammonia solution containing 0 to 50% of alkylamines selected from methylamine, diethylamine, triethylamine, and diisopropylethylamine at 55° C. for 14 hours. Reaction progress and related impurities were analyzed by HT/LCMS. As a result, the combination of 10% triethylamine and 2.0 M lithium chloride was selected for the lithium assisted cleavage and deprotection for 5′-pivaloyloxymethyl (POM) protected (E)-vinylphosphonate conjugated oligonucleotides with lower impurity profile such as PO impurity, alkylamine adduct, acrylonitrile migration adduct, and/or acrylaldehyde removal adduct. See Table 7A.

### Example 8A. Application of Lithium Assisted Cleavage and Deprotection to 5′-Phosphate Bioconjugated Oligonucleotides

1 μmol of 5′-Solid CPR II bioconjugated oligonucleotides on UnySupport CPG was dried under argon flow and treated with 10% triethylamine and 2.0 M lithium chloride in concentrated (28-30%) aqueous ammonia solution (100 to 500 μL/μmol) at 40° C. or 55° C. until the cleavage and deprotection was complete. Reaction progress and related impurities were analyzed by HT/LCMS. As a result, the cleavage and deprotection was complete within 14-18 hours at 40° C. and 10-14 hours at 55° C. See Table 8A.

### Example 9A. Stability/Efficacy Test of Lithium Assisted Cleavage and Deprotection to 5′-POM Protected (E)-Vinylphosphonate Bioconjugated Oligonucleotides

2 μmol of 5′-POM protected (E)-vinylphosphonate bioconjugated oligonucleotide on UnySupport CPG was periodically subjected to treatment with a stored solution of 2.0 M lithium chloride, 10% triethylamine in concentrated (28-30%) aqueous ammonia solution (250 μL/μmol) over a 12-month period. The solution was stored at 4° C. and room temperature (15-25° C.), each periodic treatment was at 50° C. for 14 hours in a capped/scaled container. The stability of the cleavage and deprotection solutions was verified for both quantity and quality of full-length product using Novatia high-throughput characterization system. The obtained results demonstrated that the cleavage and deprotection solution, stored for a 12-month period as described, maintained a high level of stability/efficacy through the test period without exhibiting significant changes in the purity and quantity of the oligonucleotide after the cleavage and deprotection. Sec Table 10A and Table 11A.

### Comparative Example 1A. Cleavage and Deprotection for Tri-GalNAc Bioconjugated Oligonucleotides with AMA and/or TFA Aqueous Solution

1 μmol of 5′-tri-GalNAc bioconjugated oligonucleotides on UnySupport CPG was dried under argon flow and treated with freshly made AMA solution (1:1/v of concentrated (28-30%) aqueous ammonia and 40% methylamine, 100 μL/μmol) at room temperature for 2-6 hours. Solid support was filtered off and rinsed three times with water for a total volume of 300 to 500 μL/μmol. After partial concentration to remove the excess AMA, the residue was treated with the equal volume of 5% aqueous trifluoroacetic acid to make 2.5% as final concentration and shaken at room temperature for 1-10 hours to remove UnySupport tether. Reaction mixture was quenched with triethylamine until solution reached to pH 7. Once quenched, the solution was stable at 5° C. and either purified on HPLC or desalted by diafiltration. Reaction progress and related impurities were analyzed by HT/LCMS. See Table 9A and FIG. 2e-1 and FIG. 2e-2.

TABLE 9A
Two-step cleavage and deprotection of tri-GalNAc bioconjugated oligonucleotides

| Step | Solution | Process | Cleavage | Deprotection | Follow-up |
|---|---|---|---|---|---|
| AMA | 100 μL/μmol | RT, 6 | UnySupport | Complete | Rinsing, filtering, and |
| | | hours | tether adduct | | quenching, and partial |
| | | | 88% | | evaporating |
| 5% TFA | 500 μL/μmol | RT, 10 | Complete | Complete | Quenching and further |
| | | hours | | | purificaiton |

### Comparative Example 2A. Cleavage and Deprotection for 5′-POM Protected (E)-Vinylphosphonate Conjugated Oligonucleotides with 3% DEA in Concentrated (28-30%) Ammonia Solution

1 μmol of 5′-POM protected EVP bioconjugated oligonucleotides on UnySupport CPG was dried under argon flow and treated with 3% diethylamine in concentrated (28-30%) aqueous ammonia solution (100 μL/μmol) at 55° C. for 14 hours. Reaction progress and related impurities were analyzed by HT/LCMS. As a result, cleavage and deprotection was incomplete to show little N-Acetyl protected oligonucleotide, and there were found those impurities such as PO impurity, diethylamine adduct, acrylonitrile migration adduct, and/or acrylaldehyde removal adduct. See Table 7A.

### Example 1B and 2B. Synthesis of the 3′-Tri-GalNAc Conjugated Oligonucleotides

See Table 3B. Example 1B refers to DMT-ON synthesis, and purification by oligonucleotide purification column (OPC). Example 2B refers to DMT-OFF synthesis, and purification by anion exchange chromatography (AEX). The solid phase synthesis of oligonucleotide was carried out on an Oligo Pilot 100 synthesizer (ÄKTA) using a 12 mL column. The solid support used was UnySupport 500 Å CPG (59.8 μmol/g) from ChemGenes. During synthesis, each chain elongation consisted of four steps: detritylation, coupling, oxidation/thiolation, and capping (no capping for the final cycle). Coupling of the first monomer onto the UnySupport had extra detritylation (using 10% DCA in toluene) and double capping. Final detritylation was varied on their purification method thereof. General procedure was as described below.

### Coupling Conditions for Non-Nucleosidic GalNAc Phosphoramidite on UnySupport

120 μmol of UnySupport solid support (2.0 g) was placed in synthesis column. The detritylation was performed using 10% DCA in toluene, followed by 8 CV of acetonitrile wash to remove excess detritylation reagent. The subsequent coupling step was performed by co-delivering 0.15 M non-nucleosidic GalNAc phosphoramidite solution and 1.0 M DCI activator in acetonitrile with 70% activator to amidite volumetric ratio. 2× Molar equivalents of non-nucleosidic GalNAc phosphoramidite was used. After delivering the non-nucleosidic GalNAc phosphoramidite/activator mixture, the coupling mixture was circulated via a recycling loop. Recirculation time was 25 min for non-nucleosidic GalNAc phosphoramidite. The couplings were followed by 4 CV of acetonitrile wash prior to the next step. One CV of 0.1 M CSO solution as an oxidizer was delivered at a 5× molar equivalents for a total of 5 min contact time. Following the oxidation and acetonitrile wash, capping was performed using a 1:1 (v/v) in-line mixture of Cap A (N-methylimidazole in acetonitrile, 2:8 v/v) and Cap B (acetic anhydride, 2,6-lutidine in acetonitrile, 2:3:5 v/v/v) reagents using 1.5 CV and 90 seconds contact time. This capping step was repeated twice for non-nucleosidic GalNAc phosphoramidites. If needed to conjugate several non-nucleosidic GalNAc phosphoramidite, the same synthesis cycle was repeated as described.

### Coupling Conditions for Following Non-Nucleosidic GalNAc Phosphoramidites and Nucleosidic Phosphoramidites

The detritylation was performed using 3% DCA in toluene, followed by a total of 8 CV of acetonitrile wash to remove excess detritylation reagent. The subsequent coupling step was performed by co-delivering 0.15 M phosphoramidites solution and 0.6 M ETT activator in acetonitrile with 60% activator to amidite volumetric ratio. 2× Molar equivalents of phosphoramidites was used. After delivering the phosphoramidite/activator mixture, the coupling mixture was circulated via a recycling loop. Recirculation time was 10 min for RNA couplings. The couplings were followed by a 4 CV of acetonitrile wash prior to the next step. A 50 mM iodine in pyridine and water (9:1 v/v) solution as an oxidizer was delivered at a 4× molar equivalents for a total of 4 min contact time. If the linkage was phosphorothioate, thiolation using 0.06 M DDTT in pyridine and acetonitrile (1:1; v/v) was performed at 6× molar equivalents for 6.0 min contact time. Following the oxidation or thiolation and acetonitrile wash, capping was performed using a 1:1 (v/v) in-line mixture of Cap A and Cap B reagents using 1.5 CV for 90 seconds contact time. No capping was performed for the final cycle. After completion of the final cycle, the terminal 5′-DMT group was On or Off according to the following purification method. For DMT-Off synthesis, the terminal 5′-DMT was removed by a final detritylation step. Lastly, the solid support was washed with 8 CV of acetonitrile and dried afterwards by an argon flow for a minimum of 30 min. All synthesis parameters were based on OP100 process for similar oligonucleotide sequences.

Condition for Cleavage and Deprotection of the 3′-Tri-GalNAc Conjugated oligonucleotides

After drying by argon flow, the solid support was removed from the synthesis column and transferred into a large volume tube. Solid support was treated with 12 mL/120 μmol of freshly made AMA solution (1:1 v/v of 28˜30% aqueous ammonia and 40% methylamine) and incubated at ambient temperature with 200 rpm shaking or rocking for 2˜6 hours. After reaction was complete, the solid support was filtered and washed three times with water for a total volume of 46 mL/120 μmol. The filtrate was collected. Next, the solution was partially concentrated to remove excess amount of AMA. For 3′-tri-GalNAc coupled oligonucleotide on the UnySupport there was not a removal of the UnySupport moiety with AMA deprotection, and this was seen as FLP +275 m/z or FLP +293 m/z. After drying off AMA, the residue was treated with 100 mL of 2.5% TFA per 120 μmol. This solution was placed at room temperature for 1-6 hour or 10° C. for 6-10 hour to remove all the UnySupport moiety. Next, the solution was quenched with triethylamine until solution reaches to pH 7. Once quenched, the solution was stable at 4° C. and either purified on HPLC or desalted by diafiltration. The cleavage and deprotection sample was analyzed by Hight-throughput MS, LC/MS and HPLC. Note: It was important to remove all AMA and to not introduce a buffer like TEAA before acid hydrolysis of UnySupport as it interfered with the speed of acid hydrolysis. Additionally, 5% TFA solution was tolerated when testing however increasing % TFA too much (e.g., 15% TFA) and increasing reaction time (e.g., 10-24 hours in 2.5% TFA) resulted in cleavage of GalNAc seen as FLP-202 m/z mass.

### Condition for Purification of the 3′-Tri-GalNAc Conjugated Oligonucleotides

Generally, the 3′-tri-GalNAc conjugated oligonucleotides were purified via OPC or HPLC or directly desalted if the purity was qualified over 85% of FLP. DMT-On synthesis followed the OPC purification and DMT-Off synthesis followed the HPLC purification, either via reverse-phase (RP) or anion exchange (AEX) column chromatography using a Gilson HPLC & Liquid Handler on the YMC SmartSep Q10 resin. To prepare the AEX column chromatography sample load, crude cleavage and deprotection material was partially concentrated and dissolved with Mobile Phase A until material was completely dissolved within below 15 ms/cm of conductivity and below 40 OD/mL of concentration. Sample load should then be filtered through a 0.2 μm membrane. Volume and OD quantity of sample load was recorded. The estimated crude quantity of material should be between 10,000-20,000 OD and the recommended sample loading for the YMC SmartSep Q10 resin was less than 300 OD/mL. Using a 78.5 mL column this gives a maximum sample loading of approximately 23,500 OD per prep. Sample load was split between multiple preps if loading would be more than 300 OD/mL of resin.

Note: When synthesizing the 3′-tri-GalNAc conjugated oligonucleotides at this scale using the Oligo Pilot 100, crude purity greater than 85% was achieved and so no purification was performed. The crude material was simply desalted directly after cleavage and deprotection.

### HPLC Purification Condition:

Column specification: ID×Length, 20 mm×250 mm
Column volume: 78.5 mL.
Resin: BioPro IEX SmartSep Q10
Flow rate: 16 mL/min
UV Detection: 260 nm
Temperature: 60° C.
Mobile Phase A: 15:85 (v/v) Acetonitrile and 20 mM Sodium phosphate in water
Mobile Phase B: 15:85 (v/v) Acetonitrile, 20 mM Sodium phosphate, and 1 M Sodium bromide in water

### Condition for Desalting of the 3′-Tri-GalNAc Conjugated Oligonucleotides

Desalting process was equipped with a peristaltic pump, tubing and diafiltration unit. Prior to concentration/diafiltration, the acetonitrile concentration of the purified oligonucleotide was reduced to less than 10% v/v by dilution with water. Sample reservoir was filled with purified oligonucleotide up to the 200 mL mark. The siphon port tubing was submerged in a secondary container for any volumes greater than 200 mL. Pump speed was gradually increased until a pressure of 2.5 bar, approximately pump setting 5 on peristaltic pump. A steady drip should begin to flow from the permeate line and sample should slowly begin to flow from the bulk container into the sample reservoir via the siphon tube. Once the sample had been concentrated into the sample reservoir, 500 mL of water was added to the original sample container. After 1 hour, the conductivity of the permeate was measured. If conductivity was under 100 μS/cm, then diafiltration was complete. Water feed was removed, and sample was allowed to concentrate to the desired volume. It was recommended not to concentrate the sample below 50 mL as the system had difficulty pumping low volumes and could potentially rupture the peristaltic tubing if ran dry. If conductivity was above 100 μS/cm, then diafiltration was continued. Water feed was added as necessary until conductivity was below 100 μS/cm. Concentrated sample was transferred into the final container, and system was rinsed with water for 5 minutes to drain the residual sample, which was added in final sample container. OD of the final desalted sample was measured, and the recovery data was calculated.

### Comparative Examples. Synthesis of the 3′-Tri-GalNAc Conjugated Oligonucleotides with Synthetic Variation

### Comparative Example 1B: Change of 1st Detritylation with 3% DCA in Toluene

The first detritylation of DMT protection group was removed by 3% DCA in toluene instead of 10% DCA in toluene. Other process was all the same as example 2B.

### Comparative Example 2B: Change of 1st Coupling Condition with 0.6 M ETT in Acetonitrile

The first coupling reaction was accomplished by 0.6 M ETT in Acetonitrile (70%). Other process was all the same as example 2B.

### Comparative Example 3B: Change of 1st Coupling Condition with 0.10 M GalNAc Amidite in Acetonitrile

The first coupling reaction was accomplished with 4 equivalents of 0.10 M GalNAc amidite in Acetonitrile for 25 min. Other process was all the same as example 2B.

### Comparative Example 4B: Change of 1st Oxidizing Reagent with 50 mM Iodine in Pyridine and Water

The first coupling reaction was followed by oxidation with 5 equivalents of 50 mM iodine in pyridine and water (9:1 v/v) for 5 min. Other process was all the same as example 2B.

### Comparative Example 5B: Change of 1st Capping Duration

The first coupling reaction was followed by capping with a 1:1 mixture (v/v) of Cap A (20% NMI in Acetonitrile) and CapB (acetic anhydride, 2,6-lutidine in Acetonitrile, 2:3:5 v/v/v) for 5 min. Other process was all the same as example 2B.

### Comparative Example 6B: Application of 1st Coupling Cycle Condition to Whole Process

All synthetic process was accomplished by utilizing the 1st coupling cycle condition as described in Table 3B.

### Comparative Example 7B: Application of the Following Coupling Cycle Condition to Whole Process

All synthetic processes were accomplished by utilizing the 2nd and following coupling cycle condition. Comparative example 7B utilized the multiple changes from original conditions as described in Table 3B.

TABLE 2B
Sequences information for 3'-tri-GalNAc conjugated oligonucleotides

| SEQ | | | DMT |
|---|---|---|---|
| ID NO | Sequence ID (5′→3′) | MW | Mode |
| SEQ ID | 5′-(mG)*(fC)*(mA)(fG)(fU)(fA)(mU)(fG)(mU) | 7181.1 | OFF |
| NO: 13 | (fU)(mG)(fA)(mU)(fG)(mG)(fA)(GalNAc) | | |
| | (GalNAc)(GalNAc)-3′ | | |
| SEQ ID | 5′-(mU)*(fG)*(mU)(fG)(fC)(fC)(mU)(mU) | 7011.0 | ON |
| NO: 14 | (mC)(mU)(mC)(fA)(mU)(mC)(mU)(mA)(GalNAc) | | |
| | (GalNAc)(GalNAc)-3′ | | |

TABLE 3B
Synthesis and Processing results for the synthesis variations

| | | | Example Number | | | |
|---|---|---|---|---|---|---|
| | | | | | Comparative | Comparative |
| | | | Example 1B | Example 2B | example 1B | example 2B |
| | | | Synthesis scale | | | |
| Process | | | 120 μmol | 160 μmol | 10 μmol | 10 μmol |
| Coupling | Detritylation | 10% DCA | no | no | 3% DCA | no |
| Cycle for | | in toluene | variation | variation | in toluene | variation |
| 1st GalNAc | Activator | 1.0M DCI in | no | no | no | 0.6M ETT in |
| amidite | | Acetonitrile, | variation | variation | variation | Acetonitrile, |
| on Uny- | | 70% | | | | 70% |
| Support | Amidite | 0.15M in | no | no | no | no |
| | | Acetonitrile, | variation | variation | variation | variation |
| | | 2 eq., 25 min | | | | |
| | Oxidizer | 0.1M CSO in | no | no | no | no |
| | | Acetonitrile, | variation | variation | variation | variation |
| | | 5 eq., 5 min | | | | |
| | Capping | Cap A and | no | no | no | no |
| | | Cap B (1:1 | variation | variation | variation | variation |
| | | v/v), 1.5 min | | | | |
| Coupling | Detritylation | 3% DCA in | no | no | no | no |
| Cycle for | | toluene | variation | variation | variation | variation |
| following | Activator | 0.6M ETT in | no | no | no | no |
| amidites | | Acetonitrile, | variation | variation | variation | variation |
| | | 60% | | | | |
| | Amidite | 0.15M in | no | no | no | no |
| | | Acetonitrile or | variation | variation | variation | variation |
| | | Acetonitrile/ | | | | |
| | | DCM (1:1 v/v), | | | | |
| | | 2 eq., 10 min | | | | |
| | Oxidizer | 50 mM Iodine in | no | no | no | no |
| | | Pyridine and | variation | variation | variation | variation |
| | | water (9:1 v/v), | | | | |
| | | 4 eq., 4 min | | | | |
| | Capping | Cap A and Cap B | no | no | no | no |
| | | (1:1 v/v), 1.5 min | variation | variation | variation | variation |
| | Final | 3% DCA in | DMT-On | DMT-Off | DMT-Off | DMT-Off |
| | Detritylation | toluene | synthesis | synthesis | synthesis | synthesis |
| Cleavage and | AMA | RT, 6 h | FLP: 0.1% | FLP: 2.3% | FLP: 0.9% | FLP: 1.4% |
| Deprotection* | treatment | | FLP + DMT + | FLP + Uny | FLP + Uny | FLP + Uny |
| | | | Uny-Support: | Support: | Support: | Support: |
| | | | 86.4% | 83.5% | 89.3% | 71.5% |
| | 2.5% TFA | RT, 6 h | FLP: 89.55% | FLP: 84.1% | FLP: 88.0%% | FLP: 64.2% |
| | treatment | | FLP + DMT + | FLP + Uny | FLP + Uny | FLP + Uny |
| | | | UnySupport: | Support: | Support: | Support: |
| | | | Not | Not | Not | Not |
| | | | observed | observed | observed | observed |
| Purification* | HPLC | Anion-exchange | FLP 91.5% | FLP 90.5% | Not applied | FLP 87.3% |
| | | (AEX) | | | | |
| | Desalting | Tangential flow | FLP 92.5% | FLP 90.5% | FLP 89.1% | FLP 86.0% |
| | | filtration (TFF) | | | | |

| | | | | |
|---|---|---|---|---|
| Final Yield | 55.5% | 55.8% | 27.4% | 18.1% |

| | | | Example Number | | | | |
|---|---|---|---|---|---|---|---|
| | | | Comparative | Comparative | Comparative | Comparative | Comparative |
| | | | example 3B | example 4B | example 5B | example 6B | example 7B |
| | | | Synthesis scale | | | | |
| Process | | | 10 μmol | 10 μmol | 10 μmol | 10 μmol | 10 μmol |
| Coupling | Detritylation | 10% DCA in | no | no | no | no | 3% DCA in |
| Cycle for | | toluene | variation | variation | variation | variation | toluene |
| 1st | Activator | 1.0M DCI in | no | no | no | no | 0.6M ETT in |
| GalNAc | | Acetonitrile, | variation | variation | variation | variation | Acetonitrile, |
| amidite | | 70% | | | | | 60% |
| on Uny- | Amidite | 0.15M in | 0.10M in | no | no | no | 0.15M in |
| Support | | Acetonitrile, | Acetonitrile, | variation | variation | variation | Acetonitrile or |
| | | 2 eq., 25 min | 4 eq., 25 min | | | | Acetonitrile/ |
| | | | | | | | DCM (1:1 v/v), |
| | | | | | | | 2 eq., 10 min |
| | Oxidizer | 0.1M CSO in | no | 50 mM Iodine | no | no | 50 mM Iodine |
| | | Acetonitrile, | variation | in Pyridine | variation | variation | in Pyridine and |
| | | 5 eq., 5 min | | and water | | | water (9:1 v/v), |
| | | | | (9:1 v/v), | | | 4 eq., 4 min |
| | | | | 5 eq., 5 min | | | |
| | Capping | Cap A and Cap | no | no | Cap A and Cap | no | Cap A and Cap |
| | | B (1:1 v/v), | variation | variation | B (1:1 v/v), | variation | B (1:1 v/v), |
| | | 1.5 min | | | 5 min | | 1.5 min |
| Coupling | Detritylation | 3% DCA in | no | no | no | 10% DCA in | no |
| | | toluene | variation | variation | variation | toluene | variation |
| Cycle for | Activator | 0.6M ETT in | no | no | no | 1.0M DCI in | no |
| following | | Acetonitrile, | variation | variation | variation | Acetonitrile, | variation |
| amidites | | 60% | | | | 70% | |
| | Amidite | 0.15M in | no | no | no | 0.15M in | no |
| | | Acetonitrile or | variation | variation | variation | Acetonitrile, | variation |
| | | Acetonitrile/ | | | | 2 eq., 25 min | |
| | | DCM (1:1 v/v), | | | | | |
| | | 2 eq., 10 min | | | | | |
| | Oxidizer | 50 mM Iodine | no | no | no | 0.1M CSO in | no |
| | | in Pyridine | variation | variation | variation | Acetonitrile, | variation |
| | | and water | | | | 5 eq., 5 min | |
| | | (9:1 v/v), | | | | | |
| | | 4 eq., 4 min | | | | | |
| | Capping | Cap A and Cap | no | no | no | Cap A and Cap | no |
| | | B (1:1 v/v), | variation | variation | variation | B (1:1 v/v), | variation |
| | | 1.5 min | | | | 1.5 min | |
| | Final | 3% DCA in | DMT-Off | DMT-Off | DMT-Off | DMT-Off | DMT-Off |
| | Detritylation | toluene | synthesis | synthesis | synthesis | synthesis | synthesis |
| Cleavage and | AMA | RT, 6 h | FLP: 1.1% | FLP: Not | FLP: Not | FLP: 0.1% | FLP: 2.3% |
| Deprotection* | treatment | | FLP + | observed | observed | FLP + | FLP + |
| | | | UnySupport: | FLP + | FLP + | UnySupport: | UnySupport: |
| | | | 76.5% | UnySupport: | UnySupport: | 24.2% | 12.5% |
| | | | | 19.5% | 90.0% | | |
| | 2.5% TFA | RT, 6 h | FLP: 79.1% | FLP: 16.9% | FLP: 88.2% | FLP: 21.3% | FLP: 9.7% |
| | treatment | | FLP + | FLP + | FLP + | FLP + | FLP + |
| | | | UnySupport: | UnySupport: | UnySupport: | UnySupport: | UnySupport: |
| | | | Not | Not | Not | Not | Not |
| | | | observed | observed | observed | observed | observed |
| Purification* | HPLC | Anion- | FLP 89.7% | Not applied | Not applied | Not applied | Not applied |
| | | exchange | | due to low | | due to low | due to low |
| | | (AEX) | | purity | | purity | purity |
| | Desalting | Tangential | FLP 86.9% | Not applied | FLP 89.9% | Not applied | Not applied |
| | | flow | | due to low | | due to low | due to low |
| | | filtration | | purity | | purity | purity |
| | | (TFF) | | | | | |

| | | | | | |
|---|---|---|---|---|---|
| Final Yield | 33.5% | N/A | 53.1% | N/A | N/A |

1) Cap A (20% N-methyl imidazole (NMI) in Acetonitrile) and Cap B (Acetic anhydride, 2,6-lutidine in Acetonitrile, 2:3:5 v/v/v)
2) Cleavage and deprotection result were analyzed by LCMS
3) Purification results were analyzed by AEX-HPLC

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

Furthermore, numerous references have been made to patents and printed publications throughout this specification. Each of the above-cited references and printed publications are individually incorporated herein by reference in their entirety.

1. A composition, which is an aqueous composition comprising about 0.1 to about 40% w/w of one or more of a lithium salt, and wherein the composition is at least about 50% saturated with ammonia.

2. The composition of claim 1, further comprising one or more of an alkylamine, wherein the one or more alkylamine is present at about 1% to about 40% w/w.

3. The composition of claim 1, wherein the one or more lithium salt includes a lithium cation and one or more of an anion, wherein the one or more anion has, independently, a molar mass of at least 18 g/mol.

4. The composition of claim 1, wherein the one or more of a lithium salt is a lithium fluoride, a lithium chloride, a lithium bromide, a lithium iodide, a lithium acetate, a lithium tetraborate, a lithium carbonate, a lithium sulfate, a lithium nitrate, a lithium perchlorate, or a combination thereof.

5. The composition of claim 1, wherein the one or more of a lithium salt is a lithium chloride, a lithium bromide, a lithium iodide, or a combination thereof.

6. The composition of claim 1, wherein the one or more of a lithium salt is a lithium hydroxide.

7. The composition of claim 1, wherein the one or more of a lithium salt is a lithium chloride.

8. The composition of claim 1, comprising about 0.1 M, about 0.5 M, about 1 M, about 1.5 M, about 2 M, about 2.5 M, about 3 M, about 3.5 M, about 4 M, about 4.5 M, about 5 M, about 5.5 M, or about 6 M lithium cation.

9. The composition of claim 2, wherein the one or more of an alkylamine is a primary alkylamine, a secondary alkylamine, a tertiary alkylamine, or a combination thereof.

10. The composition of claim 2, wherein the one or more of an alkylamine is trimethylamine, triethylamine, diisopropylethylamine, or a combination thereof.

11. The composition of claim 2, wherein the one or more of an alkylamine is triethylamine.

12. The composition of claim 2, comprising about 0.1% to about 20% w/w of the one or more of an alkylamine.

13. The composition of claim 2, comprising about 3% to about 15% w/w of the one or more of an alkylamine.

14. The composition of claim 2, comprising about 3% to about 10% w/w of the one or more of an alkylamine.

15. The composition of claim 1, wherein the composition comprises about 20 to about 32% w/w ammonia.

16. The composition of claim 1, wherein the composition is at least about 90% saturated with ammonia, or the composition is saturated with ammonia.

17. (canceled)

18. The composition of claim 1, selected from one of A-G:
A) an aqueous composition comprising about 0.1 M of a lithium salt, and about 20 to about 32% w/w ammonia;
B) an aqueous composition comprising about 1 M of a lithium salt, and about 20 to about 32% w/w ammonia;
C) an aqueous composition comprising about 2 M of a lithium salt, and about 20 to about 32% w/w ammonia;
D) an aqueous composition comprising about 3 M of a lithium salt, and about 20 to about 32% w/w ammonia;
E) an aqueous composition comprising about 4 M of a lithium salt, and about 20 to about 32% w/w ammonia;
F) an aqueous composition comprising about 5 M of a lithium salt, and about 20 to about 32% w/w ammonia; or
G) an aqueous composition comprising about 6 M of a lithium salt, and about 20 to about 32% w/w ammonia.

19. The composition of claim 18, wherein either the lithium salt is lithium chloride, the ammonia is present at about 28 to about 32% w/w, or the lithium salt is lithium chloride and the ammonia is present at about 28 to about 32% w/w.

20. The composition of claim 19, wherein the composition further comprises about 3% w/w diethylamine, about 10% w/w trimethylamine, about 10% w/w triethylamine, or about 40% w/w methylamine.

21. A method, comprising contacting a solid support bound molecule with the composition of claim 1.

22. (canceled)

23. A method of cleaving a molecule bound to a solid support, comprising contacting the molecule bound to the solid support with the composition of claim 1, wherein the molecule bound to the solid support is covalently bound to the solid support, and on cleavage from the solid support the molecule includes an alcohol moiety at the point of cleavage.

24. A molecule, prepared by a method that includes the method of claim 21.

25-27. (canceled)
